(19) United States
(12) Patent Application Publication
(10) Pub. No.: US 2023/0330192 A1
(43) Pub. Date: Oct. 19, 2023

(54) METHODS OF USING POLYMERIZED HUMAN SERUM ALBUMIN

(71) Applicants: Ohio State Innovation Foundation, Columbus, OH (US); THE REGENTS OF THE UNIVERSITY OF CALIFORNIA, Oakland, CA (US)

(72) Inventors: Andre PALMER, Columbus, OH (US); Pedro J. CABRALES AREVALO, La Mesa, CA (US)

(21) Appl. No.: 18/024,871

(22) PCT Filed: Sep. 6, 2021
(86) PCT No.: PCT/US2021/049186

§ 371 Date: Mar. 6, 2023

Related U.S. Application Data
(60) Provisional application No. 63/074,751, filed on Sep. 4, 2020.

(51) Int. Cl. A61K 38/38; A61P 7/00

(52) U.S. Cl. CPC A61K 38/385; A61P 7/00

(57) ABSTRACT

Disclosed are method of using compositions comprising PolyHSA to preventing, protecting and/or treating conditions such as endothelial dysfunction, and hypercytokinemia.

### CROSS-REFERENCE TO RELATED APPLICATIONS

The application claims the benefit of U.S. Provisional Application No. 63/074,751, filed Sep. 4, 2020, which is hereby incorporated herein by reference in its entirety.

### STATEMENT REGARDING FEDERALLY SPONSORED RESEARCH OR DEVELOPMENT

This invention was made with government support under grant numbers R01 HL126945, R01 EB021926, and R01 HL138116 awarded by the National Institutes of Health. The government has certain rights in the invention.

### BACKGROUND

Sepsis is aggravated by an immune response to invading microorganisms, which occasionally leads to multiple organ failure. Early infection and septic shock are characterized by circulatory abnormalities that are usually related to intravascular volume depletion, hypotensive shock and damage to the endothelial glycocalyx. These cardiovascular changes create major microcirculatory disturbances including tissue ischemia. Therefore, reperfusion following early sepsis induced ischemia is critical for the restoration of tissue metabolic homeostasis and the prevention of multiple organ dysfunction syndrome. Unfortunately, commercially available crystalloid (e.g. saline) and colloid (e.g. gelatin, dextran, hydroxyethyl starch (HES), and albumin) based solutions only provide transient benefits when infused, and impose deleterious side effects. Crystalloids require the infusion of large volumes to restore blood volume and tissue perfusion, which can result in tissue edema and concomitant tissue injury, especially during systemic inflammatory conditions. HES based colloidal solutions have been shown to induce coagulopathies and renal injury. Dextrans and gelatins can both alter hemostasis and have been shown to induce anaphylactic reactions. Human serum albumin (HSA) is a small molecular diameter protein that can extravasate from the blood vessel causing inflammation and promoting fluid filtration, and because the solution as a whole has low viscosity it does not promote tissue perfusion.

There is a need for a methods and compositions for treating systemic inflammatory conditions.

The compositions and methods disclosed herein address these and other needs.

### SUMMARY

Provided herein are methods of treating hypercytokinemia in a subject in need thereof comprising administering to the subject a therapeutically effective amount of polymerized human serum albumin (PolyHSA) to reduce circulating cytokine levels by at least 5%, such as from 5% to 70%.

In some embodiments, the hypercytokinemia is induced by an infectious agent such as influenza (e.g., H1N1 influenza or H5N1 influenza), coronavirus infection (e.g., avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza B, Parainfluenza virus, Ebola, Epstein-Barr virus, cytomegalovirus, or group A streptococcus. In some embodiments, the hypercytokinemia is associated with graft-versus-host disease.

Also described herein are methods of preventing hypercytokinemia in a subject comprising administering to the subject a therapeutically effective amount PolyHSA to reduce or prevent an increase circulating cytokine levels. In some embodiments, the subject is infected with or has been exposed to an infectious agent such as influenza (e.g., H1N1 influenza or H5N1 influenza), coronavirus infection (e.g., avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza B, Parainfluenza virus, Ebola, Epstein-Barr virus, cytomegalovirus, or group A streptococcus. In some embodiments, the subject has received or will receive transplanted cells, transplanted tissue, a transplanted organ, or any combination thereof. In some embodiments, the transplanted cells, transplanted tissue, a transplanted organ, or any combination thereof comprise an allograft or a xenograft.

Also described herein are methods of treating endothelial dysfunction in a subject comprising administering to the subject a therapeutically effective amount of PolyHSA to reduce circulating levels of a biomarker for endothelial dysfunction in the subject.

Also described herein are methods of preventing endothelial dysfunction in a subject comprising administering to the subject a therapeutically effective amount PolyHSA to reduce or prevent an increase in circulating levels of a biomarker for endothelial dysfunction in the subject. In some embodiments, the PolyHSA can be administered in an effective amount to prevent circulating levels of the biomarker for endothelial dysfunction rising above normal levels for subjects without endothelial dysfunction. In some embodiments, the biomarker for endothelial dysfunction comprises syndecan-1.

Also described herein are methods of treating endothelial dysfunction in a subject in need thereof comprising administering to the subject a therapeutically effective amount of PolyHSA to reduce endothelial barrier permeability.

Also described herein are methods of protecting endothelial tissue in a subject comprising administering to the subject a PolyHSA in a therapeutically effective amount to protect endothelial tissue from damage.

In some embodiments, the subject has a normal blood pressure. In some embodiments, the PolyHSA is administered via infusion or exchange transfusion. In some embodiments, the PolyHSA is administered via infusion. In some embodiments, the infusion comprises infusion of a volume of a composition comprising the PolyHSA, and wherein the volume comprises from 10% to 30% of the subject's total blood volume. In some embodiments, the PolyHSA is administered via exchange transfusion. In some embodiments, the exchange transfusion comprises exchange transfusion of from 5% to 50% of the subject's total blood volume with a composition comprising the PolyHSA. In some embodiments, the PolyHSA is administered in an amount effective to reduce circulating cytokine levels by at least 5%, such as from 5% to 70%. In some embodiments, the PolyHSA can be administered in a therapeutically effective amount to reduce an immune response. In some embodiments, the PolyHSA can be administered in a therapeutically effective amount to reduce the number of leukocytes adhered to endothelial tissue in the subject. In some embodiments, the PolyHSA can be administered in a therapeutically effective amount to improve vascular integrity. In some embodiments, the PolyHSA can have a molecular weight ranging from 100 kDa to 50,000 kDa, such as from 100 kDa to 500 kDa, or from 300 kDa to 500 kDa, or from 500 kDa to 750 kDa, or from 750 kDa to 1000 kDa, or from 750 kDa to 2000 kDa.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1A-1B show (1A) mean arterial pressure (MAP) and (1B) heart rate (HR) measured throughout ischemia followed by reperfusion with no topload infusion (control), infusion of HSA, or infusion of PolyHSA. Symbols indicate significance levels (*) P<0.05 and (**) P<0.01 between treatment groups at the same time point. (n=8 animals/group)

FIG. 2A-2D show microhemodynamic diameters for arterioles (2A) and venules (2B) and blood flow for arterioles (2C) and venules (2D) relative to baseline measured throughout ischemia followed by reperfusion with no topload infusion (control), infusion of HSA, or infusion of PolyHSA. FIG. 2A-2B have the same x-axis as FIG. 2C-2D. Symbols indicate significance levels (*) P<0.05, (**) P<0.01, (***) P<0.001, and (****) P<0.0001 between treatment groups at the same time point. †: P<0.05 compared to baseline conditions. (n=12 vessels/group)

FIG. 3A-3B show (3A) functional capillary density (FCD) and (3B) immobilized leukocytes throughout ischemia followed by reperfusion with no topload infusion (control), infusion of HSA, or infusion of PolyHSA. Symbols indicate significance levels (*) P<0.05, (**) P<0.01, and (***) P<0.001 between treatment groups at the same time point. †: P<0.05 compared to baseline conditions. n=6 animals/group

FIG. 4A-4B show (4A) vascular permeability of animals post-ischemia/reperfusion injury and (4B) extravasation of HSA and PolyHSA post-ischemia/reperfusion injury. Symbols indicate significance levels (*) P<0.05, (**) P<0.01, (***) P<0.001, and (****) P<0.0001 between treatment groups at the same time point. †: P<0.05 compared to baseline conditions. n=6 animals/group

FIG. 5A-5B show the number of apoptotic and necrotic cells. (5A) Number of annexin V positive and propidium iodine (PI) positive stained cells. (5B) Number of necrotic (PI+/Annexin V−), late apoptotic (PI+/Annexin V+), and early apoptotic (PI−/Annexin V+) cells for each treatment group after 24 hours of reperfusion. †: P<0.05 compared to Sham, ‡: P<0.05 compared to animals that received no topload infusion, §: P<0.05 compared to animals that were administered HSA. (n=6 animals/group).

FIG. 6 show the ischemia-reperfusion model and illustration of groups included in the study. Animals received a topload (hypervolemic, 20% blood volume, 7% body weight) of HSA (10 g/dL) or PolyHSA (10 g/dL) after ischemia. Alternatively, a third group received no hemodilution.

FIG. 7 show polymerized human serum albumin synthesis and application as a plasma substitute. (a) Human serum albumin (HSA) (1) is reacted with glutaraldehyde to form polymerized HSA (PolyHSA) (2). (b) When transfused, unmodified HSA intermingles with red blood cells (RBCs) in the RBC rich core (6) and the RBC depleted plasma layer (5). Due to its small hydrodynamic diameter, HSA is able to easily extravasate through endothelial cell-cell junctions (4) and smooth muscle cell layers (3), which results in reduced circulatory half-life and tissue edema. (c) The increased hydrodynamic diameter of PolyHSA leads to increased vascular retention and blood viscosity.

FIGS. 8A-8G show (8A-8B) show changes in systemic hemodynamics following LPS induced endotoxemia (8A) Heart rate (HR) and (8B) mean arterial pressure (MAP), with no resuscitation, infusion of HSA, or infusion of PolyHSA; (8C-8F) changes in microcirculatory hemodynamics and wall shear stress following LPS induced endotoxemia (8C) functional capillary density (FCD), (8D) arteriole diameter, (8E) blood velocity, (8F) blood flow, and (8G) arteriole wall shear stress, with no resuscitation, infusion of HSA, or infusion of PolyHSA. The shaded region on the plots indicates the period of fluid resuscitation with PolyHSA or HSA. Data are presented as mean and SD. †: P<0.05 between the PolyHSA and no resuscitation groups at the same time point. ‡: P<0.05 between the PolyHSA and HSA groups at the same time point. Symbols next to data points indicate a significant (P<0.05) difference at that time point compared to baseline conditions for (*) no resuscitation, (§) HSA, and (?) PolyHSA (n=6 animals/group).

FIGS. 9A-9H show changes in the immune response following LPS induced endotoxemia. Number of (9A) adhered and (9B) rolling leukocytes per 100 μm. Concentrations in serum of (9C) tumor necrosis factor-alpha (TNF-α), (9D) interleukin 1 alpha (IL-1α), (9E) interleukin 1 beta (IL-1β), (9F) interleukin 6 (IL-6), (9G) interleukin 10 (IL-10), and (9H) interleukin 12 (IL-12) as measured with ELISA with no resuscitation, infusion of HSA, or infusion of PolyHSA. Data are presented as mean and SD or as boxplots where whiskers indicate 95% CI and boxes indicate data quartiles. *: P<0.05 between groups, †: P<0.05 compared to baseline conditions. (n=6 animals/group).

FIGS. 10A-10C show tissue status following LPS induced endotoxemia. (10A) Number of annexin V positive and propidium iodine (P.I.) positive stained cells with no resuscitation, infusion of HSA, or infusion of PolyHSA compared to a sham. (10B) Number of necrotic (PI+/Annexin V−), late apoptotic (PI+/Annexin V+), and early apoptotic (PI−/Annexin V+) cells for each treatment group. (10C) Endothelial permeability measured via extravascular/intravascular (EV/IV) fluorescent signals from FITC-Dextran (70 kDa M.W.). A higher ratio indicates more vascular leakage. Data are presented as mean and SD or as boxplots where whiskers indicate 95% CI and boxes indicate data quartiles. †: P<0.05 compared to Sham, ‡: P<0.05 compared to animals that received no resuscitation, §: P<0.05 compared to animals that received HSA as a resuscitation fluid. *: P<0.05 between groups, ?: P<0.05 compared to baseline conditions. (n=6 animals/group)

FIGS. 11A-11D show changes in systemic hemodynamics, functional capillary density and survival following CLP induced polymicrobial sepsis. (11A) Heart rate, (11B) mean arterial pressure, (11C) functional capillary density and (11D) survival, with no resuscitation, infusion of HSA, or infusion of PolyHSA. Data are presented as mean and SD. Survival was assessed via pairwise implementation of the log-rank test. †: P<0.05 between the PolyHSA and no resuscitation groups at the same time point. ‡: P<0.05 between the PolyHSA and HSA groups at the same time point. ¶: P<0.05 between the HSA and No resuscitation groups at the same time point. Symbols next to data points indicate a significant (P<0.05) difference at that time point compared to baseline conditions for (*) no resuscitation, (§) HSA, and (?) PolyHSA. (n=6 animals/group)

FIG. 12A-12F show changes in microhemodynamics following CLP induced polymicrobial sepsis. Arteriolar (12A) and venular (12B) blood vessel diameter, arteriolar (12C) and venular (12D) blood fluid velocity, and arteriolar (12E) and venular (12F) blood flow with no resuscitation, infusion of HSA, or infusion of PolyHSA. †: P<0.05 between the PolyHSA and no resuscitation groups at the same time point. ‡. P<0.05 between the PolyHSA and HSA groups at the same time point. ¶: P<0.05 between the HSA and no resuscitation groups at the same time point. Symbols next to data points indicate a significant (p<0.05) difference at that time point compared to baseline conditions for (*) no resuscitation, (§) HSA, and (?) PolyHSA. (n=6 animals/group)

FIG. 13 shows HSA extravasates through endothelial cell-cell junctions, which impacts vascular permeability and microcirculation in sepsis. Increased hydrodynamic diameter of PolyHSA leads to increased vascular retention and blood viscosity.

FIG. 14 shows direct visualization of the glycocalyx using fluorescently labeled lectins after resuscitation from hemorrhagic shock (HS) with PolyHSA60:1 and HES (Hextend™). The fluorescent intensity profiles of lectins that bind to the disaccharides of glycosaminoglycans (GAGs) on the endothelium indicate that PolyHSA improves endothelial integrity and protects the glycocalyx compared to HES in the hamster dorsal chamber model.

FIG. 15A-15F show resuscitation from hemorrhagic shock (HS) with PolyHSA60:1, Hextend™ and HSA. HS was induced by withdrawing 50% of the blood volume (BV), HS was sustained for 60 mins, and the animal resuscitated with the test solution. Systemic and microvascular parameters were studied at baseline (BL), HS, and 60 (R60) and 90 (R90) mins after resuscitation. (mean±SD; n=6 per group). †, P<0.05 to baseline; ‡, P<0.05 to HSA; §, P<0.05 to Hextend. Abbreviations --MAP: mean arterial pressure; HR: heart rate and; CO: cardiac output.

FIG. 16 shows hemostasis after resuscitation from HS with PolyHSA60:1, Hextend™ and HSA. PolyHSA induced minimal coagulation changes compared to Sham, whereas Hextend increased clotting time, and reduced maximum clot firmness. (mean±SD; n=6 per group). †, P<0.05 to Sham; ‡, P<0.05 to HSA; §, P<0.05 to Hextend.

FIG. 17A-17D show systemic and microhemodynamics after infusion of PolyHSA60:1 or HSA during endotoxemia. Results show uncoupling between macro- and micro-hemodynamics after LPS (10 μg/kg) with volume expansion of 30% of the BV with PolyHSA60:1 or HSA. MAP (17A) and CO (17C) decreased after 6 hours. Microvascular blood flow (17B) and FCD (17D) reduced as early as 2 hours after LPS injection. At later time points, microvascular function deteriorated more than the macrohemodynamics. However, PolyHSA prevented the decoupling of systemic and microvascular parameters. (mean±SD; n=6 per group). †, P<0.05 to BL; ‡, P<0.05 to HSA; §, P<0.05 to untreated.

FIG. 18 shows microvascular permeability. PolyHSA preserved microvascular permeability. FITC conjugated Dextran 70 kDa (FITC-dextran) was injected to determine vascular permeability, measured as the ratio between intravascular (IV) and extravascular (EV) fluorescence. The increase in vascular permeability explains the increase in capillary Hct, decrease in capillary blood flow, and reduced capillary pressure. (mean±SD; n=6 per group). †, P<0.05

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of“one or more,” “at least one,” and “one or more than one.” Therefore, an element preceded by “a” or “an” does not, without more constraints, preclude the existence of additional identical elements.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of 10 percent of the given numeric value provided such a deviation does not alter the end function or result of the value. Therefore, a value of about 1% can be construed to be a range from 0.9% to 1.1%. Furthermore, a range may be construed to include the start and the end of the range. For example, a range of 10% to 20% (i.e., range of 10%-20%) can includes 10% and also includes 20%, and includes percentages in between 10% and 20%, unless explicitly stated otherwise herein.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage. In particular, the term “treatment” includes the alleviation, in part or in whole, of the symptoms of coronavirus infection (e.g., sore throat, blocked and/or runny nose, cough and/or elevated temperature associated with a common cold). Such treatment may include eradication, or slowing of population growth, of a microbial agent associated with inflammation.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of coronavirus infection in patients. However, it will be appreciated that such prevention may not be absolute, i.e., it may not prevent all such patients developing a coronavirus infection, or may only partially prevent an infection in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective’ amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n- COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

Composition

Described herein are composition including PolyHSA. In some embodiments, the composition can further include one or more pharmaceutically acceptable carriers. In some embodiments, the composition described herein can also be mixed with blood compositions, which includes whole blood, plasma, blood fractions, crystalloid solutions, or plasma expanders (PEs), or any combination thereof.

In some embodiments, suitable plasma expander can include but is not limited to hetastarch (HEXTEND® or HESPAN®), human serum albumin, dextran, or any combination thereof.

In some embodiments, the composition does not need a plasma expander. In some embodiments, the composition without a plasma expander can reduce clot formation compared to plasma expanders such as hetastarch (HEXTEND® or HESPAN®), human serum albumin, dextran, or any combination thereof.

PolyHSA

In some embodiments, the PolyHSA can have a high molecular weight of at least 100 kDa (e.g., at least 200 kDa, at least 300 kDa, at least 400 kDa, at least 500 kDa, at least 600 kDa, at least 700 kDa, at least 750 kDa, at least 800 kDa, at least 900 kDa, at least 1000 kDa, at least 1500 kDa, at least 2000 kDa, at least 2500 kDa, at least 3000 kDa, at least 3500 kDa, at least 4000 kDa, at least 4500 kDa, at least 5000 kDa, at least 5500 kDa, at least 6000 kDa, at least 6500 kDa, at least 7000 kDa, at least 7500 kDa, at least 8000 kDa, at least 8500 kDa, at least 9000 kDa, at least 9500 kDa, at least 10,000 kDa, at least 15,000 kDa, at least 20,000 kDa, at least 25,000 kDa, at least 30,000 kDa, at least 35,000 kDa, at least 40,000 kDa, or at least 45,000 kDa. In one embodiment, a majority of the PolyHSA in the composition has a high molecular weight of at least about 200 kDa, at least about 2,000 kDa, or at least about 10,000 kDa.

In some embodiments, the PolyHSA can have a high molecular weight of 50,000 kDa or less, (e.g., 40,000 kDa or less, 30,000 kDa or less, 20,000 kDa or less, 10,000 kDa or less, 5,000 kDa or less, 4,000 kDa or less, 3,000 kDa or less, 2,000 kDa or less, 1,000 kDa or less, 750 kDa or less, 500 kDa or less, 400 kDa or less, 300 kDa or less, 200 kDa or less). In some embodiments, the PolyHSA can have a molecular weight of 300 kDa or less, 500 kDa or less, 750 kDa or less.

The PolyHSA can have a high molecular weight ranging from any of the minimum values described above to any of the maximum values described above. For example, in some embodiments, the PolyHSA can range of from 100 kDa to 50,000 kDa, (e.g., from 100 kDa to 300 kDa, from 100 kDa to 500 kDa, from 100 kDa to 750 kDa, from 100 kDa to 1000 kDa, from 100 kDa to 2000 kDa, from 100 kDa to 3000 kDa, from 100 kDa to 4000 kDa, from 100 kDa to 5000 kDa, from 100 kDa to 6000 kDa, from 100 kDa to 7000 kDa, from 100 kDa to 8000 kDa, from 100 kDa to 9000 kDa, from 100 kDa to 10,000 kDa, from 100 kDa to 20,000 kDa, from 100 kDa to 30,000 kDa, from 100 kDa to 40,000 kDa, from 200 kDa to 300 kDa, from 200 kDa to 500 kDa, from 200 kDa to 600 kDa, from 200 kDa to 300 kDa, from 200 kDa to 750 kDa, from 200 kDa to 800 kDa, from 200 kDa to 1000 kDa, from 200 kDa to 2000 kDa, from 200 kDa to 3000 kDa, 200 kDa to 4000 kDa, from 200 kDa to 5000 kDa, from 200 kDa to 6000 kDa, from 200 kDa to 7000 kDa, from 200 kDa to 8000 kDa, from 200 kDa to 9000 kDa, from 200 kDa to 10,000 kDa, from 200 kDa to 20,000 kDa, from 200 kDa to 30,000 kDa, from 200 kDa to 40,000 kDa, from 200 kDa to 50,000 kDa, from 300 kDa to 500 kDa, from 300 kDa to 600 kDa, from 300 kDa to 700 kDa, from 300 kDa to 750 kDa, from 300 kDa to 800 kDa, from 300 kDa to 900 kDa, from 300 kDa to 1000 kDa, from 300 kDa to 2000 kDa, from 300 kDa to 3000 kDa, 300 kDa to 4000 kDa, from 300 kDa to 5000 kDa, from 300 kDa to 6000 kDa, from 300 kDa to 7000 kDa, from 300 kDa to 8000 kDa, from 300 kDa to 9000 kDa, from 300 kDa to 10,000 kDa, from 300 kDa to 20,000 kDa, from 300 kDa to 30,000 kDa, from 300 kDa to 40,000 kDa, from 300 kDa to 50,000 kDa, from 400 kDa to 500 kDa, from 400 kDa to 600 kDa, from 400 kDa to 700 kDa, from 400 kDa to 750 kDa, from 400 kDa to 800 kDa, from 400 kDa to 900 kDa, from 400 kDa to 1000 kDa, from 400 kDa to 2000 kDa, from 400 kDa to 3000 kDa, 400 kDa to 4000 kDa, from 400 kDa to 5000 kDa, from 400 kDa to 6000 kDa, from 400 kDa to 7000 kDa, from 400 kDa to 8000 kDa, from 400 kDa to 9000 kDa, from 400 kDa to 10,000 kDa, from 400 kDa to 20,000 kDa, from 400 kDa to 30,000 kDa, from 400 kDa to 40,000 kDa, from 400 kDa to 50,000 kDa, from 500 kDa to 750 kDa, from 500 kDa to 800 kDa, from 500 kDa to 1000 kDa, from 500 kDa to 2000 kDa, from 500 kDa to 3000 kDa, from 500 kDa to 4000 kDa, from 500 kDa to 5000 kDa, from 500 kDa to 5000 kDa, from 500 kDa to 7000 kDa, from 500 kDa to 8000 kDa, from 500 kDa to 9000 kDa, from 500 kDa to 10,000 kDa, from 500 kDa to 20,000 kDa, from 500 kDa to 30,000 kDa, from 500 kDa to 40,000 kDa, from 500 kDa to 50,000 kDa, from 600 kDa to 750 kDa, from 800 kDa to 2,000 kDa, from 800 kDa to 3,000 kDa, from 800 kDa to 4,000 kDa, from 800 kDa to 5,000 kDa, from 800 kDa to 7,000 kDa, from 800 kDa to 10,000 kDa, from 800 kDa to 20,000 kDa, from 800 kDa to 30,000 kDa, from 800 kDa to 40,000 kDa, from 800 kDa to 50,000 kDa, from 1000 kDa to 2,000 kDa, from 1000 kDa to 3,000 kDa, from 1000 kDa to 4,000 kDa, from 1000 kDa to 5,000 kDa, from 1000 kDa to 7,000 kDa, from 1000 kDa to 10,000 kDa, from 1000 kDa to 20,000 kDa, from 1000 kDa to 30,000 kDa, from 1000 kDa to 40,000 kDa, from 1000 kDa to 50,000 kDa, from 2000 kDa to 5,000 kDa, from 2000 kDa to 10,000 kDa, from 2000 kDa to 20,000 kDa, from 2000 kDa to 30,000 kDa, from 2000 kDa to 40,000 kDa, from 2000 kDa to 50,000 kDa, from 5000 kDa to 10,000 kDa, from 5000 kDa to 20,000 kDa, from 5000 kDa to 30,000 kDa, from 5000 kDa to 40,000 kDa, from 5000 kDa to 50,000 kDa, from 10,000 kDa to 20,000 kDa, from 10,000 kDa to 30,000 kDa, from 10,000 kDa to 40,000 kDa, from 10,000 kDa to 50,000 kDa, from 20,000 kDa to 30,000 kDa, from 20,000 kDa to 40,000 kDa, from 20,000 kDa to 50,000 kDa, from 30,000 kDa to 40,000 kDa, from 30,000 kDa to 50,000 kDa, or 40,000 kDa to 50,000 kDa. In some embodiments, the PolyHSA can range of from 100 kDa to 500 kDa, or 300 kDa to 500 kDa, or 500 kDa to 750 kDa, or 750 kDa to 1000 kDa, or 750 kDa to 2000 kDa.

In one embodiment, more than 50% of the PolyHSA can have a high molecular weight (MW) (i.e. MW>MW of HSA). In some embodiments, at least 50% of the PolyHSA has a high molecular weight (e.g., at least 60%, at least 70%, at least 75%, at least 80%, at least 85%, at least 90%, at least 95%).

In some embodiments, less than 100% of the PolyHSA can have a high molecular weight (e.g., less than 95%, less than 90%, less than 85%, less than 80%, less than 75%, less than 70%, at least about 65%, less than 60%, or less than 55%).

The PolyHSA can have a high molecular weight ranging from any of the minimum values described above to any of the maximum values described above. For example, in some embodiments, the PolyHSA can have a high molecular weight ranging of from 50% to 100% (e.g., from 50% to 95%, from 50% to 90%, from 50% to 85%, from 50% to 80%, from 50% to 75%, from 50% to 70%, from 50% to 65%, from 50% to 60%, from 50% to 55%, from 60% to 95%, from 60% to 90%, from 60% to 85%, from 60% to 80%, from 60% to 75%, from 60% to 70%, from 60% to 65%, from 70% to 95%, from 70% to 90%, from 70% to 85%, from 70% to 80%, from 70% to 75%, from 80% to 95%, from 80% to 90%, from 80% to 85%, or from 90% to 95%).

In some embodiments, the PolyHSA can have a cross-linker to HSA molar ratio, referred to herein as the cross-link density, of at least 10:1, (e.g., at least 20:1, at least 30:1, at least 40:1, at least 50:1, at least 60:1, at least 70:1, at least 80:1, at least 90:1). In some embodiments, the PolyHSA can have a cross-linker to HSA molar ratio, referred to herein as the cross-link density, of 100:1 or less, (e.g., 90:1 or less, 80:1 or less, 70:1 or less, 60:1 or less, 50:1 or less, 40:1 or less, 30:1 or less, 20:1 or less), and may optionally range between any of these cross-linking densities.

The PolyHSA can have a cross-linker to HSA molar ratio, referred to herein as the cross-link density, ranging from any of the minimum values described above to any of the maximum values described above. For example, the cross-linking density can be in a range from 10:1 to 100:1 (e.g., from 10:1 to 20:1, from 10:1 to 30:1, from 10:1 to 40:1, from 10:1 to 50:1, from 10:1 to 60:1, from 10:1 to 70:1, from 10:1 to 80:1, from 10:1 to 90:1, from 20:1 to 30:1, from 20:1 to 40:1, from 20:1 to 50:1, from 20:1 to 60:1, from 20:1 to 70:1, from 20:1 to 80:1, from 20:1 to 90:1, from 20:1 to 100:1, from 30:1 to 40:1, from 30:1 to 50:1, from 30:1 to 60:1, from 30:1 to 70:1, from 30:1 to 80:1, from 30:1 to 90:1, from 30:1 to 100:1, from 40:1 to 50:1, from 40:1 to 60:1, from 40:1 to 70:1, from 40:1 to 80:1, from 40:1 to 90:1, from 40:1 to 100:1, from 50:1 to 60:1, from 50:1 to 70:1, from 50:1 to 80:1, from 50:1 to 90:1, from 50:1 to 100:1, 60:1 to 70:1, from 60:1 to 80:1, from 60:1 to 90:1, from 60:1 to 100:1, from 70:1 to 80:1, from 70:1 to 90:1, from 70:1 to 100:1, from 80:1 to 90:1, from 80:1 to 100:1, from 90:1 to 100:1). The molecular weight and/or cross-link density of the PolyHSA compositions affect their biophysical characteristics, which directly determine viscosity and colloid osmotic pressure. As shown in the examples below, high MW PolyHSA compositions having higher cross-link densities generally have improved biophysical characteristics relative to native HSA and dextran.

The PolyHSA compositions have a higher viscosity than monomeric HSA compositions, when formulated at the same protein concentration. In one embodiment, the viscosity of the PolyHSA composition is 1.1 times greater than the viscosity of the monomeric HSA composition having the same concentration (e.g., 2 times greater, 3 times greater, 4 times greater, 5 times greater, 6 times greater, 7 times greater, 8 times greater, 9 times greater, or 10 times greater).

In some embodiments, the PolyHSA compositions have a lower COP than monomeric compositions at the same concentration level. In one embodiment, the COP of the PolyHSA composition can be ½ the COP of monomeric HSA (e.g., ⅓, ¼, ⅕, ⅙, 1/7, ⅛, 1/9, 1/10, 1/15, 1/20, 1/25, 1/30, 1/35, 1/40, 1/45, or 1/50 the COP of monomeric HSA). In another embodiment, the COP of the PolyHSA composition is about 1/10 the COP of monomeric HSA. In another embodiment, the COP is about 1/50 the COP of monomeric HSA.

In some embodiments, the PolyHSA may have a molecular weight of at least 100 kDa and a cross-link density of at least 10:1. In some embodiments, the PolyHSA may have a molecular weight of at least 200 kDa and a cross-link density of at least 10:1. In some embodiments, the PolyHSA may have a molecular weight of at least 300 kDa and a cross-link density of at least 10:1. In some embodiments, the PolyHSA may have a molecular weight of at least 400 kDa and a cross-link density of at least 10:1. In some embodiments, the PolyHSA may have a molecular weight of at least 500 kDa and a cross-link density of at least 10:1. In some embodiments, the PolyHSA may have a molecular weight of at least 100 kDa and a cross-link density of at least 25:1. In some embodiments, the PolyHSA may have a molecular weight of at least 200 kDa and a cross-link density of at least 25:1. In some embodiments, the PolyHSA may have a molecular weight of at least 300 kDa and a cross-link density of at least 25:1. In some embodiments, the PolyHSA may have a molecular weight of at least 400 kDa and a cross-link density of at least 25:1. In some embodiments, the PolyHSA may have a molecular weight of at least 500 kDa and a cross-link density of at least 25:1. In some embodiments, the PolyHSA may have a molecular weight of at least 100 kDa and a cross-link density of at least 50:1. In some embodiments, the PolyHSA may have a molecular weight of at least 200 kDa and a cross-link density of at least about 50:1. In some embodiments, the PolyHSA may have a molecular weight of at least 300 kDa and a cross-link density of at least 50:1. In some embodiments, the PolyHSA may have a molecular weight of at least 400 kDa and a cross-link density of at least 50:1. In some embodiments, the PolyHSA may have a molecular weight of at least 500 kDa and a cross-link density of at least 50:1. In some embodiments, the PolyHSA may have a molecular weight of at least 100 kDa and a cross-link density of at least 75:1. In some embodiments, the PolyHSA may have a molecular weight of at least 200 kDa and a cross-link density of at least 75:1. In some embodiments, the PolyHSA may have a molecular weight of at least 300 kDa and a cross-link density of at least 75:1. In some embodiments, the PolyHSA may have a molecular weight of at least 400 kDa and a cross-link density of at least 75:1. In some embodiments, the PolyHSA may have a molecular weight of at least 500 kDa and a cross-link density of at least 75:1. In some embodiments, the PolyHSA may have a molecular weight of at least 100 kDa and a cross-link density of at least 100:1. In some embodiments, the PolyHSA may have a molecular weight of at least 200 kDa and a cross-link density of at least 100:1. In some embodiments, the PolyHSA may have a molecular weight of at least about 300 kDa and a cross-link density of at least 100:1. In some embodiments, the PolyHSA may have a molecular weight of at least 400 kDa and a cross-link density of at least 100:1. In some embodiments, the PolyHSA may have a molecular weight of at least 500 kDa and a cross-link density of at least 100:1.

In some embodiments, the PolyHSA can be made by polymerizing monomeric HSA with a cross-linker, quenching the polymerization reaction with a reducing agent, and collecting the PolyHSA having the desired molecular weight.

Suitable monomeric HSA can come from any source such as HSA isolated from human serum using known techniques or recombinant HSA. The monomeric HSA is diluted or concentrated to the desired level, such as to 25 mg/mL with a suitable buffer. The polymerization reaction is initiated by the addition of a cross-linker, such as a 70% glutaraldehyde solution, to the HSA solution at the desired molar ratio of cross-linker to HSA: such as at least 10:1, at least 50:1, and at least 100:1. The cross-linking density of the resulting PolyHSA composition may be controlled by controlling this molar ratio or by controlling the parameters of the polymerization reaction, such as the duration and temperature of the reaction. The cross-link density of a PolyHSA composition can be confirmed by separating PolyHSA from any free cross-linker after the polymerization reaction and quantifying the amount of free cross-linker compared to the initial amount of cross-linker used in the reaction. The difference between the two quantities would be equivalent to the amount of cross-linker that is cross-linked to the protein. Glutaraldehyde, like many cross-linkers, reacts with lysine, histidine, tyrosine, arginine, and primary amine groups, forming both intra and intermolecular cross-links within HSA and between neighboring HSA molecules in solution. Therefore, cross-linked HSA compositions can include polymers of various molecular weights.

Suitable cross-linkers in addition to glutaraldehyde can include succindialdehyde, activated forms of polyoxyethylene and dextran, α-hydroxy aldehydes, such as glycolaldehyde, N-maleimido-6-aminocaproyl-(2′-nitro,4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylaminopropyl)carbodiimide hydrochloride, N,N′-phenylene dimaleimide, and compounds belonging to the bisimidate class, the acyl diazide class or the aryl dihalide class, and combinations thereof.

The HSA is allowed to polymerize with the cross-linker for a suitable period of time to obtain HSA having the desired MW. For example, the polymerization reaction may be incubated at about 37° C. for between 1 and 4 hours. The polymerization reaction is then quenched with a molar excess of reducing agent, preferably a strong reducing agent that is capable of reducing the Schiff bases in the PolyHSA and any remaining free aldehyde groups on the cross-linker. For example, the reaction may be quenched by incubating the reaction mixture with a 1 M sodium borohydride solution for 30 min at 37° C. Quenching the Schiff bases in the PolyHSA stabilizes the polymer and prevents the hydrolysis of PolyHSA back to monomeric HSA, which could extravasate and cause detrimental side effects. Moreover, reducing the aldehyde group on the cross-linker completely quenches the polymerization reaction. An exemplary strong reducing agent capable for use in embodiments of the invention is sodium borohydride, however it is understood that other reducing agents may be useful as well.

The MW distribution of the PolyHSA in the quenched reaction mixture will be affected by the conditions under which the polymerization reaction is conducted, such as duration and temperature of the incubation along with the cross-linker to HSA molar ratio. To control for variables in the polymerization reaction that might result in PolyHSA having a MW outside of the desired range, the process further includes the step of collecting PolyHSA having the desired MW range. The collecting step may include separating or purifying PolyHSA having the desired MW range or making the PolyHSA free from undesirable elements such as HSA having a MW outside of the desired range. For example, the PolyHSA solution may be clarified such as by being passed through a glass chromatography column packed with glass wool to remove large particles. The clarified PolyHSA solution is then separated into distinct molecular mass fractions using known separation methods such as passing the clarified PolyHSA solution through a tangential flow filtration (TFF) hollow fiber (HF) cartridge selected to collect PolyHSA having the desired MW. For example, fractionation of the PolyHSA composition with a 100 kDa TFF HF cartridge (Spectrum Labs, Rancho Dominguez, Calif.) will result in the retentate containing PolyHSA molecules that are at least 100 kDa or larger and that fall within the desired MW in one embodiment of the invention. In that example, the filtrate will mostly contain PolyHSA molecules that are smaller than 100 kDa, i.e., molecules that are smaller than the desired MW. The MW of the PolyHSA can be controlled by passing the clarified PolyHSA solution through TFF HF cartridges having different pore sizes selective for the desired MW.

The PolyHSA solution may then be subjected to as many cycles of diafiltration with an appropriate buffer as needed in order to remove impurities having a MW outside of the desired range. The PolyHSA solution may also buffer exchanged to remove impurities such as unpolymerized cross-linkers and quenching agents which may be cytotoxic. After separation of the desired fraction, the filtrate may subsequently be concentrated such as with a 100 kDa TFF HF cartridge (Spectrum Labs). The MW distribution of the PolyHSA may be confirmed by known methods such as SDS-PAGE analysis or size exclusion chromatography coupled with multi-angle static light scattering.

Methods of Use

Described herein are methods of treating hypercytokinemia in a subject in need thereof comprising administering to the subject a therapeutically effective amount of PolyHSA to reduce circulating cytokine levels by at least 5% compared to an untreated subject (e.g., at least 10%, at least 15%, at least 25%, at least 30%, at least 35%, at least 40%, at least 45%, at least 50%, at least 55%, at least 60%, or at least 65%). In some embodiments, the method comprising administering to the subject a therapeutically effective amount of PolyHSA to reduce circulating cytokine levels by 70% or less compared to an untreated subject, (e.g., 65% or less, 60%6 or less, 55% or less, 50% or less, 45% or less, 40% or less, 35% or less, 30% or less, or 25% or less, or 20% or less, or 15% or less, or 10% or less).

The method comprising administering to the subject a therapeutically effective amount of PolyHSA to reduce circulating cytokine levels can range from any of the minimum values described above to any of the maximum values described above. For example, the method comprising administering to the subject a therapeutically effective amount of PolyHSA to reduce circulating cytokine levels can range from 5% to 70% compared to an untreated subject, (e.g., from 5% to 60%, from 5% to 50%, from 5% to 40%, 5% to 30%, from 5% to 20%, from 5% to 10%, from 10% to 70%, from 10% to 60%, from 10% to 50%, from 10% to 40%, 10% to 30%, from 10% to 20%, 20% to 60%, from 20% to 50%, from 20% to 40%, 20% to 30%, from 30% to 70%, from 30% to 60%, from 30% to 50%, from 30% to 40%, from 40% to 70%, from 40% to 60%, from 40% to 50%, from 50% to 70%, from 50% to 60%, or from 60% to 70%).

The hypercytokinemia can be associated with an infectious or non-infectious etiology. For example, in some embodiments, the hypercytokinemia can be induced by an infectious agent such as influenza (e.g., H1N1 influenza or H5N1 influenza), coronavirus infection (e.g., avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza B, Parainfluenza virus, Ebola, Epstein-Barr virus, cytomegalovirus, or group A streptococcus. The hypercytokinemia can also be associated with a non-infectious condition such as graft-versus-host disease.

Described herein are also methods of preventing hypercytokinemia in a subject comprising administering to the subject a therapeutically effective amount of PolyHSA to reduce or prevent an increase circulating cytokine levels. In some embodiments, the subject is infected with or has been exposed to an infectious agent such as influenza (e.g., H1N1 influenza or H5N1 influenza), coronavirus infection (e.g., avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza B, Parainfluenza virus, Ebola, Epstein-Barr virus, cytomegalovirus, or group A streptococcus. In some embodiments, the subject has received or will receive transplanted cells, transplanted tissue, a transplanted organ, or any combination thereof. In some embodiments, the transplanted cells, transplanted tissue, a transplanted organ, or any combination thereof comprise an allograft or a xenograft.

Also described herein are methods of preventing, protecting and/or treating endothelial dysfunction comprising administering to the subject a therapeutically effective amount of PolyHSA to reduce circulating levels of a biomarker for endothelial dysfunction in the subject.

In some embodiments, described herein are methods of treating endothelial dysfunction in a subject comprising administering to the subject a therapeutically effective amount of PolyHSA to reduce circulating levels of a biomarker for endothelial dysfunction in the subject. In some embodiments, described herein are methods of treating endothelial dysfunction in a subject comprising administering to the subject a therapeutically effective amount PolyHSA to reduce or prevent an increase in circulating levels of a biomarker for endothelial dysfunction in the subject.

In some embodiments, the PolyHSA can be administered in an effective amount to prevent circulating levels of the biomarker for endothelial dysfunction rising above normal levels for subjects without endothelial dysfunction. In some embodiments, circulating biomarkers for endothelial dysfunction can include but are not limited to angiopoietin-1, angiopoietin-2, syndecan-1, von Willebrand factor (vWF), thrombomodulin, thrombospondin-2, circulating endothelial cells (CEC) and circulating endothelial progenitor cells (or in general CEC expressing the membrane glycoprotein CD146), E-selectin (selectin family expressed on the surface of endothelial cells), ICAM-1 and VCAM-1 (endothelial ligands for leukocytes and platelets), and endothelial microparticles (EMP). In some embodiments, the circulating biomarkers for endothelial dysfunction van be syndecan-1. Circulating biomarkers for endothelial dysfunction can be measured from a blood sample.

In some embodiments, the methods of preventing, protecting and/or treating endothelial dysfunction can include administering to the subject a therapeutically effective amount of PolyHSA to reduce endothelial barrier permeability by at least at least 5%, (e.g., at least 10%, at least 20%, at least 35%, at least 40%, at least 45%, at least 50%, at least 55%, at least 60%, or at least 65%). In some embodiments, the methods of preventing, protecting and/or treating endothelial dysfunction can include administering to the subject a therapeutically effective amount of PolyHSA to reduce endothelial barrier permeability by 70% or less, (e.g., 65% or less, 60% or less, 55% or less, 50% or less, 45% or less, 40% or less, 35% or less, 20% or less, or 10% or less). The methods of preventing, protecting and/or treating endothelial dysfunction can include administering to the subject a therapeutically effective amount of PolyHSA to reduce endothelial barrier permeability ranging from any of the minimum values described above to any of the maximum values described above. For example, the methods of preventing, protecting and/or treating endothelial dysfunction can include administering to the subject a therapeutically effective amount of PolyHSA to reduce endothelial barrier permeability can range from 5% to 70%, (e.g., from 5% to 60%, from 5% to 50%, from 5% to 40%, 5% to 30%, from 5% to 20%, from 5% to 10%, from 10% to 70%, from 10% to 60%, from 10% to 50%, from 10% to 40%, 10% to 30%, from 10% to 20%, 20% to 60%, from 20% to 50%, from 20% to 40%, 20% to 30%, from 30% to 70%, from 30% to 60%, from 30% to 50%, from 30% to 40%, from 40% to 70%, from 40% to 60%, from 40% to 50%, from 50% to 70%, from 50% to 600%, or from 600% to 70%).

In some embodiments, the endothelial barrier permeability is reduced compared to the endothelial barrier permeability in a subject not treated with the composition described herein. In some embodiments, the endothelial barrier permeability is reduced compared to the endothelial barrier permeability in the subject prior to treatment with the composition described herein. In some embodiments, the composition prevents the endothelial barrier permeability.

In some embodiments, the methods of preventing, protecting and/or treating endothelial dysfunction can include administering to the subject a therapeutically effective amount of PolyHSA to protect endothelial tissue from damage. In some embodiments, the methods of preventing, protecting and/or treating endothelial dysfunction can include administering to the subject a therapeutically effective amount of PolyHSA to reduce inflammatory immune response by at least 5% (e.g., at least 10%, at least 25%, at least 30%, at least 35%, at least 40%, at least 45%, at least 50%, at least 55%, at least 60%, or at least 65%). In some embodiments, the methods of preventing, protecting and/or treating endothelial dysfunction can include administering to the subject a therapeutically effective amount of PolyHSA to reduce inflammatory immune response by 70% or less (e.g., 65% or less, 60% or less, 55% or less, 50% or less, 45% or less, 40% or less, 35% or less, 30% or less, or 25% or less, 15% or less, or 10% or less).

The methods of preventing, protecting and/or treating endothelial dysfunction can include administering to the subject a therapeutically effective amount of PolyHSA to reduce inflammatory immune response can range from any of the minimum values described above to any of the maximum values described above. For example, the methods of preventing, protecting and/or treating endothelial dysfunction can include administering to the subject a therapeutically effective amount of PolyHSA to reduce inflammatory immune response can range from 5% to 70%, (e.g., from 5% to 60%, from 5% to 50%, from 5% to 40%, 5% to 30%, from 5% to 20%, from 5% to 10%, from 10% to 70%, from 10% to 60%, from 10% to 50%, from 10% to 40%, 10% to 30%, from 10% to 20%, 20% to 60%, from 20% to 50%, from 20% to 40%, 20% to 30%, from 30% to 70%, from 30% to 60%, from 30% to 50%, from 30% to 40%, from 40% to 70%, from 40% to 60%, from 40% to 50%, from 50% to 70%, from 50% to 60%, or from 60% to 70%). In some embodiments, the inflammatory immune response is reduced compared to the inflammatory immune response in a subject not treated with the composition described herein. In some embodiments, the inflammatory immune response is reduced compared to the inflammatory immune response in the subject prior to treatment with the composition described herein. In some embodiments, the composition prevents an increase in circulating biomarkers for endothelial dysfunction, which can be measured from a blood sample, comprise angiopoietin-1, angiopoietin-2, syndecan-1, vWF, thrombomodulin, thrombospondin-2, CEC, E-selectin, ICAM-1 and VCAM-1, and/or EMP.

In some embodiments, the methods of preventing, protecting and/or treating endothelial dysfunction can include administering to the subject a therapeutically effective amount of PolyHSA to reduce the number of leukocytes adhered to an endothelial tissue by at least 5%, (e.g., at least 10%, at least 15%, at least 25%, at least 30%, at least 35%, at least 40%, at least 45%, at least 50%, at least 55%, at least 60%, at least 65%, at least 70%, or at least 75%). In some embodiments, the methods of preventing, protecting and/or treating endothelial dysfunction can include administering to the subject a therapeutically effective amount of PolyHSA to reduce the number of leukocytes adhered to an endothelial tissue by 80% or less, (e.g., 75% or less, 70% or less, 65% or less, 60% or less, 55% or less, 50% or less, 45% or less, 40% or less, 35% or less, 30% or less, 25% or less, 20% or less, or 10% or less).

The methods of preventing, protecting and/or treating endothelial dysfunction can include administering to the subject a therapeutically effective amount of PolyHSA to reduce the number of leukocytes adhered to an endothelial tissue can range from any of the minimum values described above to any of the maximum values described above. For example, the methods of preventing, protecting and/or treating endothelial dysfunction can include administering to the subject a therapeutically effective amount of PolyHSA to reduce the number of leukocytes adhered to an endothelial tissue can range from 5% to 80%, (e.g., from 5% to 70%, from 5% to 60%, from 5% to 50%, from 5% to 40%, 5% to 30%, from 5% to 20% from 5% to 10%, from 10% to 80%, from 10% to 70%, from 10% to 60%, from 10% to 50%, from 10% to 40%, from 10% to 30%, from 100% to 20% from 20% to 80%, from 20% to 70%, from 20% to 60%, from 20% to 50%, from 20% to 40%, 20% to 30%, from 30% to 80%, from 30% to 70%, from 30% to 60%, from 30% to 50%, from 30% to 40%, from 40% to 70%, from 40% to 60%, from 40% to 50%, from 50% to 80%, from 50% to 70%, from 50% to 60%, from 60% to 80%, from 60% to 70%, or from 70% to 80%). In some embodiments, the number of leukocytes adhered to an endothelial tissue is reduced compared to the number of leukocytes adhered to an endothelial tissue in a subject not treated with the composition described herein. In some embodiments, the number of leukocytes adhered to an endothelial tissue is reduced compared to the number of leukocytes adhered to an endothelial tissue in the subject prior to treatment with the composition described herein. In some embodiments, the composition prevents an increase in number of leukocytes adhered to an endothelial tissue. Changes in leukocyte activation can be measured from circulating biomarkers for endothelial surface selectins including E-selectin, ICAM-1 and VCAM-1.

In some embodiments, the preventing, protecting and/or treating endothelial dysfunction can include administering to the subject a therapeutically effective amount of PolyHSA to improve vascular integrity by at least at least 5%, (e.g., at least 10%, at least 20%, at least 35%, at least 40%, at least 45%, at least 50%, at least 55%, at least 60%, or at least 65%). In some embodiments, the preventing, protecting and/or treating endothelial dysfunction can include administering to the subject a therapeutically effective amount of PolyHSA to improve vascular integrity by 70% or less, (e.g., 65% or less, 60% or less, 55% or less, 50% or less, 45% or less, 40% or less, 35% or less, 20% or less, or 10% or less).

The preventing, protecting and/or treating endothelial dysfunction can include administering to the subject a therapeutically effective amount of PolyHSA to improve vascular integrity ranging from any of the minimum values described above to any of the maximum values described above. For example, the preventing, protecting and/or treating endothelial dysfunction can include administering to the subject a therapeutically effective amount of PolyHSA to improve vascular integrity can range from 5% to 70%, (e.g., from 5% to 60%, from 5% to 50%, from 5% to 40%, 5% to 30%, from 5% to 20%, from 5% to 10%, from 10% to 70%, from 10% to 60%, from 10% to 50%, from 10% to 40%, 10% to 30%, from 10% to 20%, 20% to 60%, from 20% to 50%, from 20% to 40%, 20% to 30%, from 30% to 70%, from 30% to 60%, from 30% to 50%, from 30% to 40%, from 40% to 70%, from 40% to 60%, from 40% to 50%, from 50% to 70%, from 50% to 60%, or from 60% to 70%). In some embodiments, the vascular integrity is improved compared to the vascular integrity in a subject not treated with the composition described herein. In some embodiments, the vascular integrity is improved compared to the improve vascular integrity in the subject prior to treatment with the composition described herein. Improvements in vascular integrity can be measured by a reduction or normalization of circulating biomarkers for endothelial dysfunction, which can be measured from a blood sample, comprise angiopoietin-1, angiopoietin-2, syndecan-1, vWF, thrombomodulin, thrombospondin-2, CEC, E-selectin, ICAM-1 and VCAM-1, and/or EMP.

In some embodiments, the subject can have a normal pressure of circulating blood on the walls of blood vessels (“normal blood pressure”) based on the subjects age, gender, posture, and exercise state. In some embodiments, the subject can have a low pressure of circulating blood on the walls of blood vessels (“low blood pressure”). Normal blood pressure levels are known in the art and may vary slightly depending on the subject's age, gender, posture, and exercise state.

In some embodiments, the compositions described herein can be administered in an effective amount to reduce extravasation through the endothelium, resulting in the maintenance of intravascular oncotic pressure. In some embodiments, the administration of compositions described herein can stabilize intravascular oncotic pressure for a period of at least 24 hours.

In some embodiments, the compositions described herein can also be useful to treat clinical conditions hypercytokinemia, inflammatory immune response, endothelial dysfunction, multiorgan dysfunction syndrome, endotoxemia, sepsis or combinations thereof. The compositions described herein could be used to treat vascular leakage due to inflammation and fibrosis such as diabetes, chronic inflammation, brain edema, arthritis, uvietis, macular edema, cancer, hyperglycemia, a kidney inflammatory disease, a disorder resulting in kidney fibrosis, a disorder of the kidney resulting in proteinuria, sepsis, or combinations thereof. PolyHSA's ability to preserve the glycocalyx may also indicate its use as an agent to increase plasma viscosity (and thus endothelial shear stress) following ischemia, such as after surgery, stroke, myocardial infarction, or extended tourniquet application, or during states of hypercoagulability, in order to prevent the microvascular dysfunction that frequently occurs due to ischemia injury.

In some embodiments, the conditions mentioned may be caused by an infectious agent such as influenza (e.g., H1N1 influenza or H5N1 influenza), coronavirus infection (e.g., avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza B, Parainfluenza virus, Ebola, Epstein-Barr virus, cytomegalovirus, or group A streptococcus.

Methods of Administration

The compositions as used in the methods described herein can be administered by any suitable method and technique presently or prospectively known to those skilled in the art. For example, the active components described herein can be formulated in a physiologically- or pharmaceutically acceptable form. In some embodiments, the composition described herein can be administered via infusion or exchange transfusion into the circulatory system of a subject, such as intravenous or intraarterial through a catheter.

In some embodiments, the composition described herein can also be mixed with blood compositions, which includes whole blood, plasma, blood fractions, crystalloid solutions, or plasma expanders (PEs), or any combination thereof prior to infusion or exchange transfusion into the subject.

Compositions, as described herein, comprising an active compound (e.g., PolyHSA) and an excipient of some sort may be useful in a variety of medical and non-medical applications.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, camauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

In some embodiments, the PolyHSA is administered via infusion or exchange transfusion. In some embodiments, the PolylHSA is administered via infusion. In some embodiments, the PolyHSA is administered via exchange transfusion

In some embodiments, when the composition described herein is administered via infusion the volume varies depending on the condition in the subject. In some embodiments, the infusion includes infusion of a volume of a composition comprising the PolyHSA, and wherein the volume includes at least 10%/o of the subject's total blood volume (e.g., at least 15%, at least 20%, at least 25%). In some embodiments, the infusion includes infusion of a volume of a composition comprising the PolyHSA, and wherein the volume includes 30% or less of the subject's total blood volume (e.g., 25% or less, 20% or less, or 15% or less).

The infusion includes infusion of a volume of a composition comprising the PolyHSA, and wherein the volume includes a range from any of the minimum values described above to any of the maximum values described above. For example, In some embodiments, the infusion includes infusion of a volume of a composition comprising the PolyHSA, and wherein the volume includes a range from 10% to 30% of the subject's total blood volume (e.g., from 10% to 25%, from 10% to 20%, from 10% to 15%, from 15% to 20%, from 15% to 25%, from 15% to 30%, from 20% to 25%, from 20% to 30%, or from 25% to 30%). In some embodiments, the infusion includes infusion of a volume of a composition comprising the PolyHSA, and wherein the volume includes from 10% to 30% of the subject's total blood volume.

In some embodiments, when the composition described herein is administered via exchange transfusion the exchanged transfusion volume varies depending on the condition in the subject. In some embodiments, the exchange transfusion includes exchange transfusion of least 5% of the subject's total blood volume with a composition comprising the PolyHSA (e.g., at least 10%, at least 15%, at least 20%, at least 25%, at least 30%, at least 35%, at least 40%, or at least 45%). In some embodiments, the exchange transfusion includes exchange transfusion of 50% or less of the subject's total blood volume with a composition comprising the PolyHSA (e.g., 45% or less, 40% or less, 35% or less, 30% or less, 25% or less, 20% or less, 15% or less, or 10% or less). In some embodiments, the exchange transfusion includes exchange transfusion of 50% or less of the subject's total blood volume with a composition comprising the PolyHSA.

The exchange transfusion includes exchange transfusion ranging from any of the minimum values described above to any of the maximum values described above. For example, in some embodiments, the exchange transfusion includes exchange transfusion ranging from 5% to 50% of the subject's total blood volume with a composition comprising the PolyHSA (e.g., from 5% to 45%, from 5% to 40%, from 5% to 35%, from 5% to 30%, from 5% to 25%, from 5% to 20%, from 5% to 15%, from 5% to 10%, from 10% to 50%, from 10% to 45%, from 10% to 40%, from 10% to 35%, from 10% to 30%, from 10% to 25%, from 10% to 20%, from 10% to 15%, from 15% to 20%, from 15% to 25%, from 15% to 30%, from 15% to 35%, from 15% to 40%, from 15% to 45%, from 15% to 50%, from 20% to 25%, from 20% to 30%, from 25% to 30%, from 25% to 35%, from 25% to 40%, from 25% to 45%, from 25% to 50%, from 30% to 35%, from 30% to 40% from 30% to 45%, from 30% to 50%, from 35% to 40%, from 35% to 45%, from 35% to 50%, from 40% to 45%, from 40% to 50%, or from 45% to 50%). In some embodiments, the exchange transfusion includes exchange transfusion of from 5% to 50% of the subject's total blood volume with a composition comprising the PolyHSA.

Administration of the compositions can be a single administration, or at continuous and distinct intervals as can be readily determined by a person skilled in the art.

The composition may be administered in such amounts, time, and route deemed necessary in order to achieve the desired result. The exact amount of the active ingredient (e.g., PolyHSA) will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications. Dosage can vary, and can be administered in one or more dose administrations daily, for one or several days.

In some embodiments, the compound as used in the methods described herein may be administered in combination or alternation with one or more additional active agents. Representative examples additional active agents include antimicrobial agents (including antibiotics, antiviral agents and anti-fungal agents), anti-inflammatory agents (including steroids and non-steroidal anti-inflammatory agents), anti-coagulant agents, immunomodulatory agents, anticytokine, antiplatelet agents, and antiseptic agents.

Representative examples of antibiotics include amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, and vancomycin.

Representative examples of antiviral agents include, but are not limited to, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, and zidovudine.

Representative examples of anticoagulant agents include, but are not limited to, heparin, warfarin, rivaroxaban, dabigatran, apixaban, edoxaban, enoxaparin, and fondaparinux.

Representative examples of antiplatelet agents include, but are not limited to, clopidogrel, ticagrelor, prasugrel, dipyridamole, dipyridamole/aspirin, ticlopidine, and eptifibatide.

Representative examples of antifungal agents include, but are not limited to, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, and amphotericin b.

Representative examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Representative examples of non-steroidal anti-inflammatory drugs include ibuprofen, naproxen, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

Other examples of additional active agents include chloroquine, hydrochloroquine, Vitamin D, and Vitamin C.

Reference will now be made in detail to specific aspects of the disclosed materials, compounds, compositions, articles, and methods, examples of which are illustrated in the accompanying Examples and Figures.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

By way of non-limiting illustration, examples of certain embodiments of the present disclosure are given below.

### EXAMPLES

Sepsis is aggravated by an immune response to invading microorganisms, which occasionally leads to multiple organ failure. Early infection and septic shock are characterized by circulatory abnormalities that are usually related to intravascular volume depletion, hypotensive shock and damage to the endothelial glycocalyx. These cardiovascular changes create major microcirculatory disturbances including tissue ischemia. Therefore, reperfusion following early sepsis induced ischemia is critical for the restoration of tissue metabolic homeostasis and the prevention of multiple organ dysfunction syndrome. Unfortunately, commercially available crystalloid (e.g. saline) and colloid (e.g. gelatin, dextran, hydroxyethyl starch (HES), and albumin) based solutions only provide transient benefits when infused, and impose deleterious side effects. Crystalloids require the infusion of large volumes to restore blood volume and tissue perfusion, which can result in tissue edema and concomitant tissue injury, especially during systemic inflammatory conditions. HES based colloidal solutions have been shown to induce coagulopathies and renal injury. Dextrans and gelatins can both alter hemostasis and have been shown to induce anaphylactic reactions. Human serum albumin (HSA) is a small molecular diameter protein that can extravasate from the blood vessel causing inflammation and promoting fluid filtration, and because the solution as a whole has low viscosity it does not promote tissue perfusion. Despite these drawbacks, HSA is generally safe, and increasing its molecular size can mitigate its side effects. A simple and cost-effective strategy to increase the molecular size of HSA is via polymerization using the non-specific cross-linking agent glutaraldehyde, which polymerizes HSA molecules to generate polymerized HSA (PolyHSA).

PolyHSA possesses significantly larger molecular diameter compared to HSA, which prevents its extravasation especially during states of inflammation (characterized by decreased osmotic pressure), which prevents sudden shifts in fluid volume, and its higher viscosity promotes vascular recovery following hypoperfusion. Since PolyHSA does not extravasate into the tissue space, the colloidal osmotic pressure it exerts is restricted solely to the intravascular space, which prevents vascular leakage and promotes fluid reabsorption at the capillary level, ultimately stabilizing blood volume and the endothelial glycocalyx.

Additionally, fluid reabsorption decreases tissue hydrostatic pressure, which promotes capillary recruitment and tissue perfusion, preventing the deleterious buildup of metabolic byproducts that exacerbate inflammation. The steric hinderance of the HSA molecules in PolyHSA also prevents the extravasation of toxic molecules (heme, bilirubin, transition metals) into the tissue space, which has less oxidative protection than plasma; simultaneously, this maintains intravascular concentrations of drugs that bind with HSA (ibuprofen, propofol) which prevents adverse shifts in these drugs' pharmacokinetics during systemic inflammation and local ischemia.

PolyHSA's decreased osmotic pressure relative to unmodified HSA prevents sudden shifts in fluid volume. Sudden shifts in fluid volume exacerbate ischemia and reperfusion injury, and ultimately cause additional inflammation by driving sudden bursts of oxygen, which trigger the production of hydroxyl radicals and initiates an inflammatory cascade, feeding a positive feedback loop that can drive additional inflammation through glycocalyx deterioration and protein extravasation. By smoothing shifts in fluid volume following ischemia, PolyHSA prevents this feedback loop. Additionally, PolyHSA-based solutions have higher viscosity than HSA-based solutions. Increased viscosity increases endothelial shear stress, which is well known to promote autacoid production and improve tissue perfusion. However, increased endothelial shear stress also promotes the production of glycosaminoglycans (GAGs), which form the glycocalyx. By promoting GAG production, increased viscosity therefore also promotes endothelial glycocalyx stability. Promoting glycocalyx stability through shear stress mediated mechanotransduction also prevents vascular leakage and inflammation, as the negatively charged glycocalyx repels native colloidal proteins.

### Example 1: Attenuating Ischemia and Reperfusion Injury with Polymerized Albumin

Abstract

Increased vascular permeability following reperfusion of ischemic tissue results in extravasation of fluid and small proteins from the intravascular compartment into the tissue space, leading to increased interstitial fluid pressure and capillary collapse, impairing capillary exchange and eliciting tissue damage. We hypothesize that the infusion of a polymerized human serum albumin (PolyHSA) molecule with increased molecular weight (MW) compared to unpolymerized human serum albumin (HSA) may improve vascular fluid retention, which can improve recovery from ischemia-reperfusion injury. In this prospective study, we evaluated how the infusion of PolyHSA immediately before reperfusion of local ischemic tissue impacts microhemodynamics, vascular integrity, and tissue viability in a hamster dorsal window chamber model. Microvascular flow and functional capillary density were maintained in animals exchanged with PolyHSA. In the reperfused tissue, exchange with PolyHSA preserved vascular permeability measured with extravasation of fluorescently labeled dextran. Analysis of tissue viability indicated that exchange with PolyHSA reduced the number of apoptotic cells 24 hours after reperfusion. Maintenance of microvascular perfusion, improvement in vascular integrity, and reduction in tissue damage resulting from reperfusion with PolyHSA indicates that PolyHSA is a promising fluid therapy for improving outcomes of ischemia-reperfusion injury.

Introduction

Reperfusion injury is encountered when treating a wide variety of clinical scenarios related to stroke¹, cardiopulmonary bypass², surgery, transplantation³⁻⁵, coronary angioplasty⁶, and thrombolytic therapy⁷. The blood supply deficit during ischemia leads to tissue damage due to lack of oxygen, and subsequent shift to anaerobic metabolism, ATP depletion, and altered ion transport⁸. In prolonged ischemia, hypoxanthine is formed as a breakdown product of ATP metabolism, and xanthine dehydrogenase is converted to the radial-producing xanthine oxidase due to the low availability of oxygen and hypoxic stress.

Upon reperfusion, the high availability of hypoxanthine and sudden availability of oxygen drives the oxidation of hypoxanthine into xanthine, which results in molecular oxygen being converted into highly reactive superoxide, hydroxyl radicals, and uric acid⁹. These radicals and reactive oxygen species attack cell membrane lipids, proteins, and glycosaminoglycans, causing further damage. They may also initiate specific biological processes by redox signaling. Ultimately, reperfusion alters microvascular flow and endothelium integrity, which results in tissue reperfusion edema^{8,10}, and severely slows recovery from the initial ischemic insult. Increased permeability through the damaged endothelium increases extravasation of small colloidal proteins such as serum albumin. Increased transport of these proteins past the endothelial barrier elevates the relative extravascular osmotic pressure, which can result in capillary collapse, impaired transvascular transport, and eventual necrosis¹¹. Thus, restoration of perfusion with the appropriate fluid that maintains perfusion and washes out metabolic byproducts is vital to rescue ischemic tissues.

Previous studies have used hydroxyethyl starch (HES), gelatin, and dextran based resuscitation fluids to improve recovery from ischemia-reperfusion injury^{12,13}. However, reperfusion with a HES solution resulted in increased edema in a hemorrhagic shock model¹⁴. Also, the US Food and Drug Administration has recently vetoed the use of HES solutions due to serious adverse effects such as unexpected coagulopathies and renal injury^{15,16}. Alternatively, perfluorocarbon solutions are a promising candidate for fluid therapy during ischemia-reperfusion¹⁷. However, perfluorocarbon solutions could emphasize ROS production during ischemia-reperfusion, and promote endothelial dysfunction and leukocyte adhesion, ultimately delaying recovery post-reperfusion. In cases of repeated ischemia-reperfusion cycles, perfluorocarbons trapped in the ischemic region could exacerbate the reperfusion injury¹⁸.

Another alternative material, glutaraldehyde polymerized human serum albumin (PolyHSA), is a promising resuscitation fluid due to its increased molecular diameter and low production cost. Unlike unmodified HSA, the increased molecular diameter of Poly HSA restricts extravasation from the intravascular space into the tissue space, which improves intravascular retention and microvascular hemodynamics¹⁹. PolyHSA also possesses increased viscosity, which promotes endothelial shear stress that may contribute to maintenance of endothelial cell mechanotransduction, glycosaminoglycan production, and ultimately endothelial glycocalyx integrity²⁰.

We hypothesize that the increased molecular size of PolyHSA should result in improved maintenance of macro- and micro-circulatory hemodynamics during ischemia-reperfusion. We sought to investigate the microcirculatory response to the administration of PolyHSA during reperfusion following ischemia. Ischemia was induced in an unanesthetized Golden Syrian hamster window chamber model. Intravital microscopy was used to assess microvascular hemodynamics and functional capillary density (FCD). To assess the impact of PolyHSA therapies on the tissue, we measured cell death in the tissue space and leukocyte-endothelial interactions.

Results

Systemic Hemodynamics

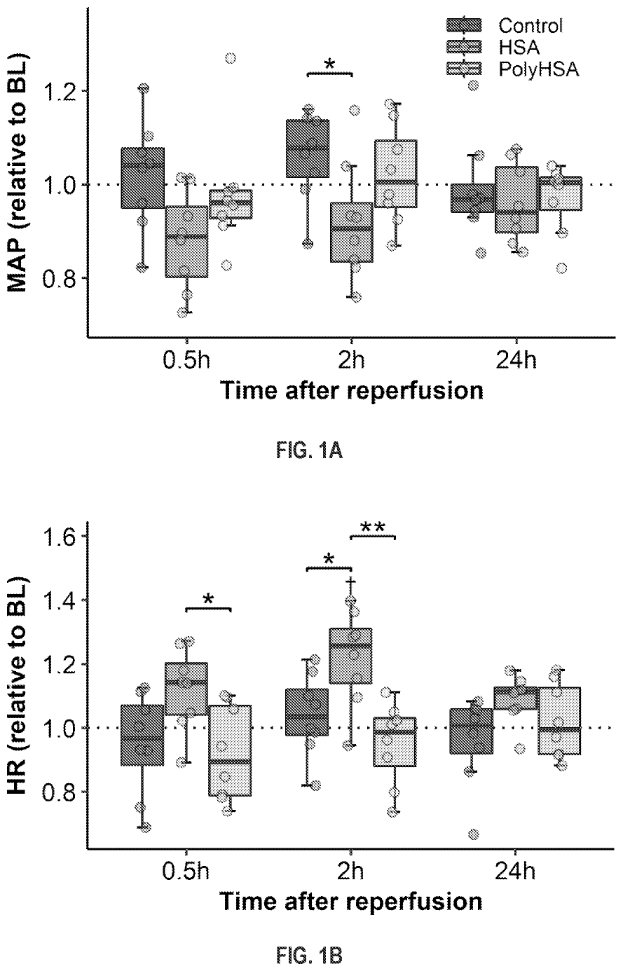

The MAP and HR measured throughout the study are shown in FIG. 1. 2 hours following reperfusion, there was a significant (P<0.05) decrease in MAP for animals infused with HSA compared to the control group. At 0.5 and 24 hours into reperfusion, there was no significant difference in MAP between each treatment group. Likewise, at 0.5 hours, there was a significant (P<0.05) increase in HR for animals infused with HSA compared to animals that were administered PolyHSA prior to reperfusion. 2 hours into reperfusion, animals administered with HSA had significantly (P<0.05) increased HR compared to the other groups.

Microcirculatory Hemodynamics

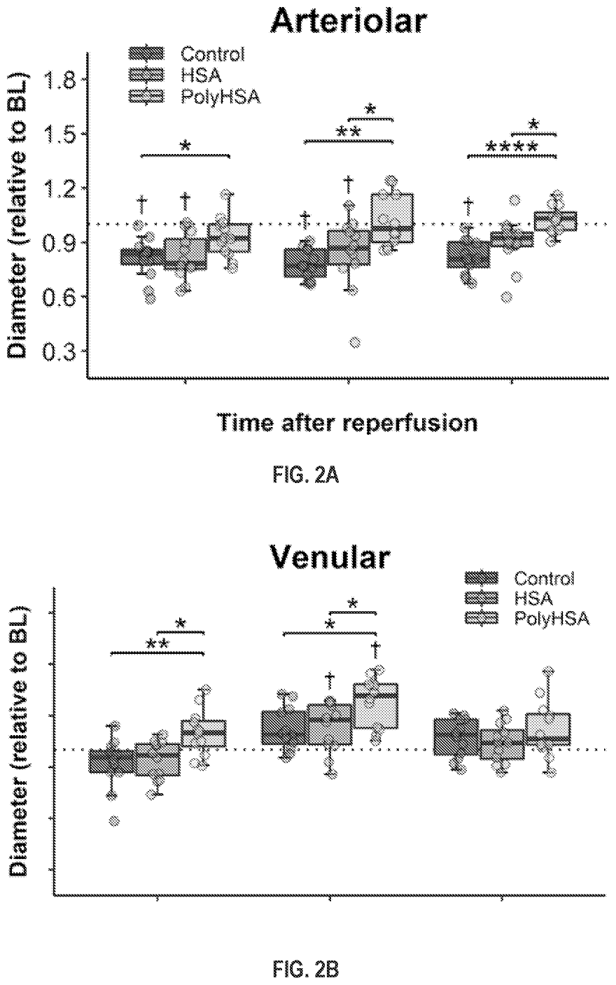

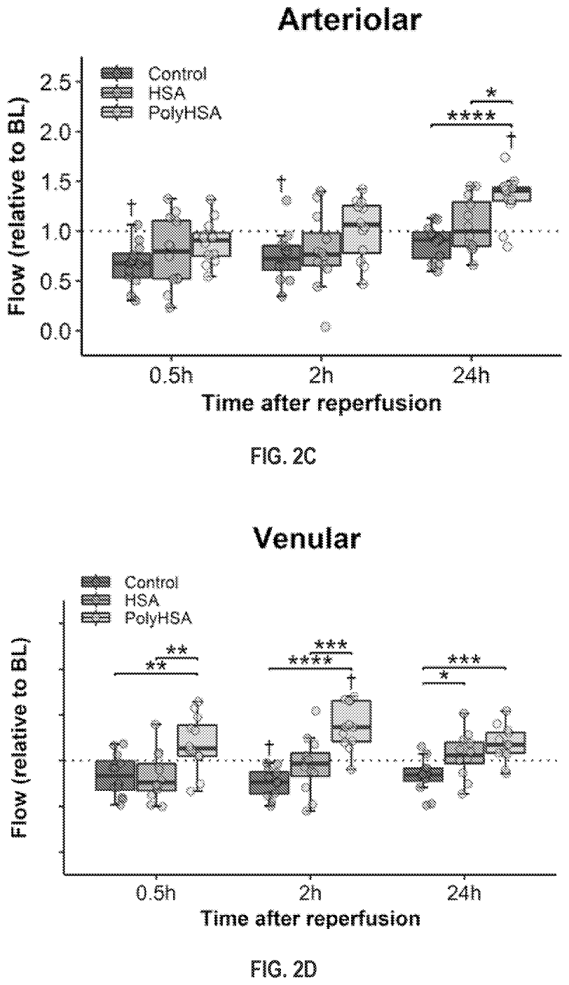

Changes in arteriolar and venular vessel diameters and blood flows measured within the chamber window model are shown in FIG. 2. Throughout reperfusion, arteriolar vessel diameters were significantly (P<0.05) wider compared to arteriolar vessel diameters in control group. For animals in the control group, arteriolar diameters were significantly (P<0.05) less than baseline conditions throughout reperfusion. At 2 and 24 hours following reperfusion, arteriolar diameters of animals that were infused with HSA were significantly (P<0.05) smaller compared to arteriolar vessels in animals infused with PolyHSA. While the other groups had significant changes in arteriolar diameters compared to baseline, arteriolar vessel diameters in animals infused with PolyHSA were not statistically different from baseline conditions throughout reperfusion. 24 hours following reperfusion, animals treated with PolyHSA had significantly (P<0.05) greater arteriolar blood flow compared to the other treatment groups at the same time point. In addition, this arteriolar flow rate was significantly (P<0.05) higher than baseline conditions. At 0.5 and 2 hours into reperfusion, arteriolar blood flow in the control group was significantly (P<0.05) lower when compared to baseline conditions. At 0.5 and 2 hours into reperfusion, venular diameters in animals infused with PolyHSA were significantly (P<0.05) larger than the HSA and control treatment groups. At two hours into reperfusion, venular flow rate of animals infused with HSA or PolyHSA was significantly greater than venular flow rates under baseline conditions. Animals infused with PolyHSA had significantly (P<0.05) greater relative venular blood flow reperfusion compared to other treatment groups throughout reperfusion. At 2 hours into reperfusion, venular blood flow in animals infused with PolyHSA was significantly (P<0.05) greater than baseline venular blood flows.

Functional Capillary Density

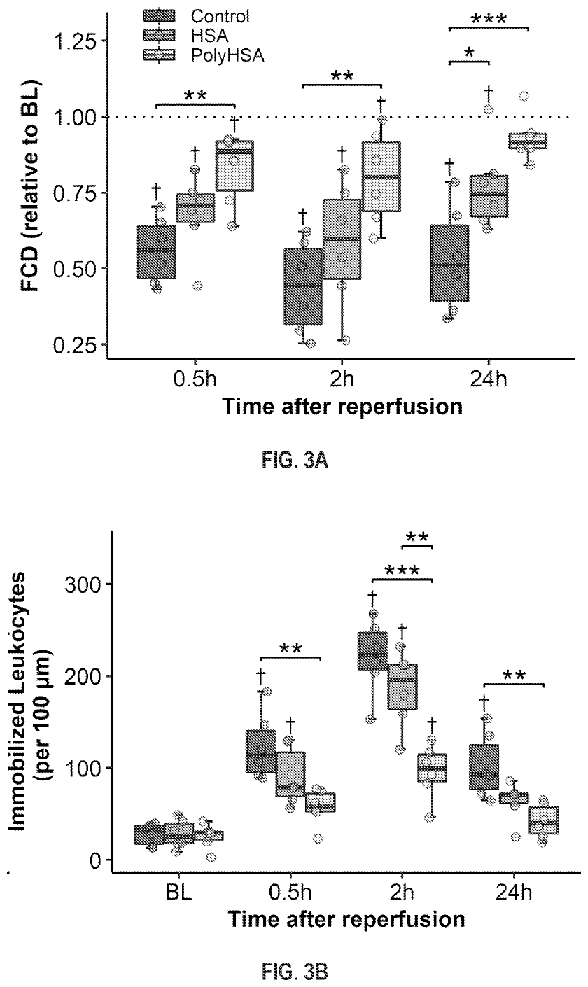

Changes in FCD during reperfusion are shown in FIG. 3a. At 0.5 and 2 hours into reperfusion, FCD of all treatment groups significantly (P<0.05) decreased compared to baseline conditions. Animals in the control group had significantly (P<0.01) decreased FCD compared to animals infused with PolyHSA throughout reperfusion. Animals treated with PolyHSA had significantly (P<0.05) increased FCD compared to all other treatment groups at 24 hours following reperfusion. While animals in the HSA and control treatment groups had significantly reduced (P<0.05) FCD compared to baseline conditions, there were no significant differences in FCD for animals infused with PolyHSA at 24 hours following reperfusion.

Leukocyte Endothelial Interactions

Changes in the number of immobilized leukocytes following ischemia and reperfusion are shown in FIG. 3b. At baseline conditions, there was no significant difference between the number of immobilized leukocytes in each treatment group. Animals in the control group had a significantly (P<0.01) greater number of immobilized leukocytes compared to animals that were infused with PolyHSA. At 2 hours into reperfusion, animals infused with PolyHSA had significantly (P<0.05) less immobilized leukocytes compared to animals in the HSA treatment group. At 2 hours, all groups had significantly (P<0.05) more immobilized leukocytes compared to baseline conditions. Throughout reperfusion, animals in the control group had significantly (P<0.05) more immobilized leukocytes compared to baseline conditions. Although animals infused with HSA had significantly (P<0.05) more immobilized leukocytes at 0.5 and 2 hours into reperfusion, there were no significant increases at 24 hours into reperfusion.

Vascular Permeability and Extravasation

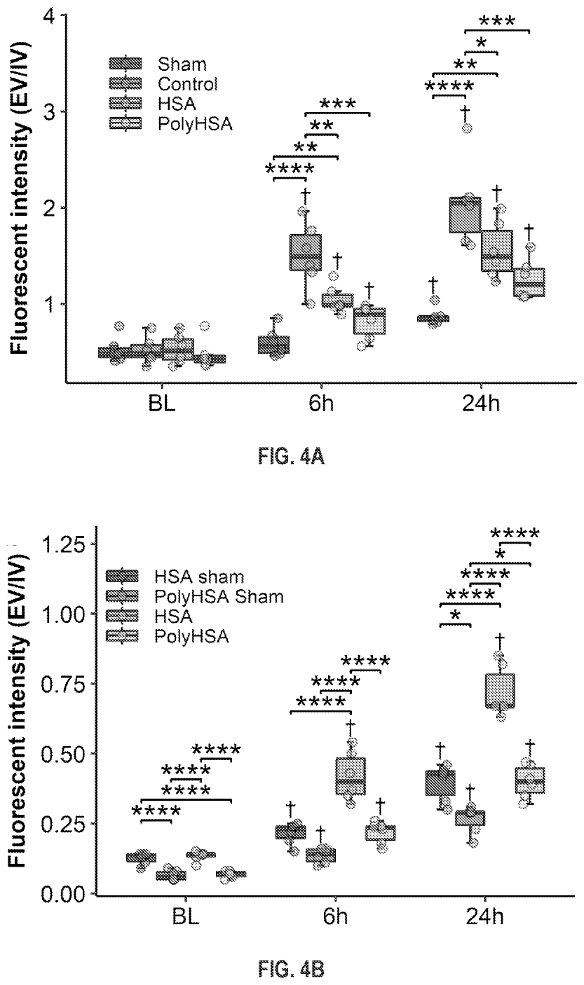

Changes in vascular permeability following ischemia and reperfusion is shown in FIG. 4a. The extravascular fluorescent intensity increased significantly (P<0.05) for all animals over the 24 hour period, indicating that the Texas Red-Dextran is not completely constrained within the vascular lumen even in Sham animals. However, control animals, and animals given a hypervolemic infusion of unmodified HSA show significantly (P<0.01) higher vascular permeability than both Sham animals and animals given a hypervolemic infusion of PolyHSA 6 h and 24 h after ischemia-reperfusion. In fact, there were no significant differences in vascular permeability between the PolyHSA and Sham groups, indicating that PolyHSA preserved vascular integrity and prevented extravasation of Texas Red-Dextran. In addition to measuring vascular permeability, we measured the extravasation of the resuscitation material itself, by conjugating HSA or PolyHSA with FITC; extravasation of the resuscitation fluid is shown in FIG. 4b. At baseline, significantly more HSA extravasated than PolyHSA (P<0.0001). Extravascular fluorescent intensity increased for all groups after 6 and 24 hours compared to baseline, indicating some passive extravasation of both HSA and PolyHSA. Ischemia-reperfusion significantly increased (P<0.05) extravasation after 24 hours for both HSA and PolyHSA compared to their respective Shams (animals not subjected to ischemia-reperfusion). Additionally, PolyHSA extravasated in animals subjected to ischemia-reperfusion to a similar extent as unmodified HSA in Sham animals over the 24 h period.

Tissue Viability

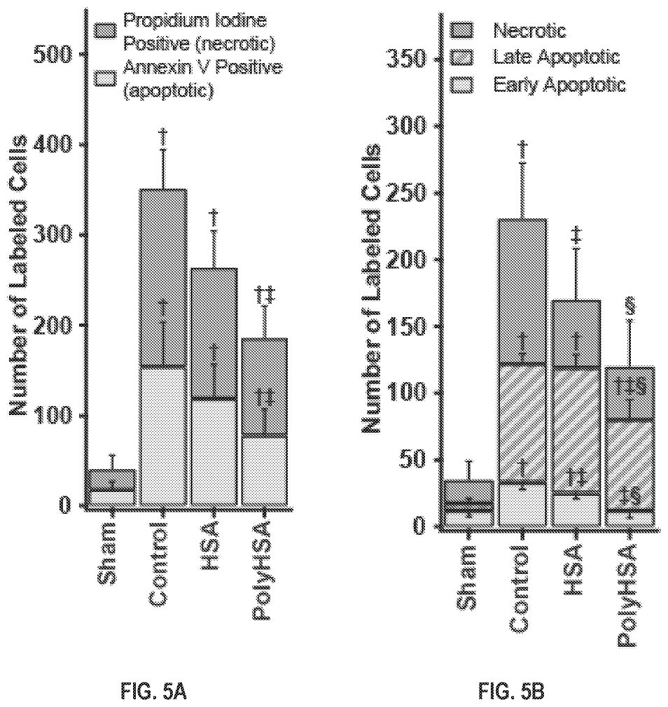

The number of apoptotic and necrotic cells after ischemia followed by 24 hours of reperfusion is shown in FIG. 5. In general, ischemia-reperfusion led to significant (P<0.05) increases in the total number of apoptotic (Annexin V+) and necrotic (PI+) cells in the tissue compared to the Sham group. In animals infused with PolyHSA there was a significant (P<0.05) decrease in the number of apoptotic and necrotic cells compared to animals in the control group. In general, ischemia-reperfusion led to significant (P<0.05) increases in the number of late apoptotic cells compared to the Sham. However, the number of early and late apoptotic cells in the tissue of animals infused with PolyHSA was significantly (P<0.05) lower than animals in the control and HSA treatment groups. In animals treated with PolyHSA, there were no significant differences in the number of early apoptotic cells compared to the Sham.

Discussion

The principal finding of this study was that intravenous topload infusion (hypervolemic, 20% blood volume over 10 minutes) of PolyHSA before reperfusion substantially mitigates reperfusion injury in an intact hamster dorsal window chamber model. When compared to an infusion of HSA, PolyHSA significantly improved maintenance of systemic hemodynamics. In the microcirculation, reperfusion with PolyHSA leads to substantially improved blood flow and maintenance of vascular tone when compared to animals reperfused with HSA or animals that received no additional fluid. PolyHSA was also significantly less prone to extravasation than HSA, and reperfusion with PolyHSA prevented an increase in vascular permeability that was observed in untreated animals and animals infused with unmodified HSA. PolyHSA induced improvements in microcirculatory hemodynamics and functional capillary density, and leads to significantly less damage to tissues. The significant decreases in the number of early apoptotic cells indicate that tissue in the ischemic zone recover from ischemia-reperfusion injury after reperfusion with PolyHSA. Because the number of late apoptotic and necrotic cells in the tissue is consistent across all treatment groups, the increase in necrotic cells is likely the result of the ischemic period.

In untreated animals, there were no significant changes in systemic parameters. This is anticipated given that ischemia was localized in this study. However, we found that infusion of unmodified HSA led to dramatic differences in systemic hemodynamics. This decrease in MAP and spike in HR is consistent with the performance of HSA in a previous hemodilution model²⁶, and is likely due to plasma volume expansion beyond the topload, stemming from HSA's high COP. PolyHSA transfusion avoided these sudden shifts in plasma volume due to its substantially lower COP, but PolyHSA normalized tissue perfusion, as its larger molecular size maintained intravascular COP. Despite having significant shifts in systemic parameters, animals infused with HSA had similar changes in microcirculatory hemodynamics compared to animals that received no topload infusion.

Animals infused with PolyHSA had a similar microcirculatory response when compared to animals administered with a perfluorocarbon (PFC) emulsion¹⁷. This similarity in results between the two solutions indicates that the mitigation of reperfusion injury observed with PFC solutions is likely unrelated to PFC facilitated oxygen transport, and could be explained by its increased viscosity, which increases vascular shear stress that can promote autacoid production, and endothelial glycocalyx stability²⁰. The PolyHSA used in these studies possessed significantly higher viscosity than native plasma or unmodified HSA, and as such, its transfusion increased plasma viscosity post-reperfusion. Additionally, PolyHSA's increased molecular diameter compared to native HSA prevented its extravasation, and thus helped maintain intravascular oncotic pressure and flow. Combined, these effects likely improved vascular endothelial shear stress and thus glycocalyx integrity in the PolyHSA group.

The effect of PolyHSA infusion on microvascular flow and FCD is comparable to the beneficial effects of colloidal expanders such as HES²⁷, whereas HSA infusion had similar effects compared to low MW dextran solution^{28,29}. This is likely due to the relatively small size of both dextran (70 kDa) and HSA (67 kDa). Unfortunately, increasing the size of dextrans results in coagulopathies, which is the primary factor that led to clinical withdrawal of HES as a resuscitation fluid³⁰, and thus likely also precludes high MW dextrans from clinical use.

The ratio of extravascular/intravascular fluorescence of Texas-red dextran confirms that glycocalyx integrity is better preserved in animals transfused with Poly HSA compared to untreated or HSA-treated animals. This resulted in a decreased immune response in the microcirculation, as evidenced by the reduction in the number of adhered leukocytes, and may have other implications. For example, the glycocalyx plays several critical roles, one of which is the prevention of thrombus formation and leukocyte endothelial interaction. Glycocalyx shedding releases heparan sulfates, which can directly activate platelets³¹. Furthermore, degradation of the glycocalyx reveals previously sterically shielded adhesion molecules, promoting thrombus attachment in injured areas, and reducing tissue perfusion³². This causes a positive feedback loop that exacerbates ischemia/reperfusion injury due to a constant state of no-flow and reflow. In disease conditions that also cause coagulopathies, particularly those driven by fibrin clotting rather than platelet aggregation such as COVID-19^{33,34}, prevention of endothelial glycocalyx degradation may have a disease-modifying effect by reducing the incidence and severity of the ischemia-reperfusion injury.

This study only had a 1-hour ischemia window before reperfusion. However, previous studies have shown that prolonged ischemia shifts the inflammatory response within the tissue⁸. These shifts can impact recovery and subsequent reperfusion-induced tissue injury. Future studies evaluating PolyHSA fluid therapy should also consider extended ischemia windows prior to reperfusion. Additionally, the parameters measured in this study only indirectly indicate the status of the endothelial glycocalyx. Future studies should measure changes in plasma levels of glycocalyx constituents, such as heparan sulfate and syndecan-1, or measure changes in glycocalyx thickness by infusing fluorescently labeled lectins that bind to the glycocalyx, to better understand the role of glycocalyx disruption in ischemia-reperfusion injury.

Conclusions

In summary, this study demonstrated a proof-of-concept application of PolyHSA in the treatment of ischemia-reperfusion injury. Compared to HSA, the increased molecular diameter of PolyHSA had a substantial impact on maintaining microvascular hemodynamics and vascular integrity, and reducing leukocyte response during reperfusion.

Methods

PolyHSA Synthesis and Analysis

HSA (ABO Pharmaceuticals, San Diego, CA) was incubated with glutaraldehyde at a 30:1 molar ratio of glutaraldehyde to HSA for 3 hours at 37° C.²¹. The reaction was quenched with sodium borohydride (NaBH₄). After the reaction, the resulting PolyHSA was diafiltered into a modified Ringer's lactate solution (115 mM NaCl, 4 mM KCl, 1.4 mM CaCl₂, 13 mM NaOH, 27 mM sodium lactate, and 2 g/L N-acetyl-L-cysteine) on a 100 kDa polysulfone hollow fiber filter (Spectrum Labs, Rancho Dominguez, CA) for four cycles. All PolyHSA solutions were filtered through a 0.2 μm filter. Both the PolyHSA and HSA solutions were prepared to a final concentration of 10 g/dL prior to infusion. The viscosity of unmodified HSA and PolyHSA solutions was measured with a DV-II+ cone and plate viscometer (Brookfield Engineering Laboratories, Middleboro, MA) at a shear rate of 160 s⁻¹ with a concentration of 10 g/dL. The PolyHSA had a higher viscosity (4.2 cP) compared to unmodified HSA (1.5 cP). The COP of the solutions was measured with a 4420 membrane colloid osmometer (Wescor, Logan, UT). The polymerization of HSA resulted in decreased COP (18 mm Hg) compared to unmodified HSA (42 mm Hg). The polymerized HSA had increased MW (410 kDa) compared to unmodified HSA (67 kDa).

Animal Preparation

All the procedures, including animal handling and care, followed the National Institutes of Health (NIH) Guide for the Care and Use of Laboratory Animals. The UC San Diego Institutional Animal Care and Use Committee approved the experimental protocol. 55-65 g Syrian Golden hamsters (Charles River Laboratories) fitted with a dorsal window chamber model were used as previously described¹⁷. The hamster window chamber model is widely used to evaluate microvascular responses in unanesthetized animals. In brief, animals were anesthetized with 50 mg/kg intraperitoneal injection of sodium pentobarbital. Sutures were used to lift the dorsal skin away from the animal. The skinfold was held between two titanium frames containing a 12 mm diameter circular hole for visualization. The skin on one side of the fold was then removed. The exposed skin was then covered with a cover glass held in place by the chamber frame. After two days, animals were anesthetized and heparinized (30 IU/mL) PE-50 catheters were inserted in the carotid artery and jugular vein. Following implantation of catheters, animals were given at least two days to recover before experiments were performed.

Inclusion Criteria

Three to four days after the final operation, systemic parameters and status of the microcirculation were examined. Animals were only considered suitable for experiments if heart rate (HR)>340 bpm, mean arterial pressure (MAP)>80 mm Hg, and microscopic examination of tissue within the chamber window showed no sign of edema or bleeding.

Ischemia-Reperfusion Protocol

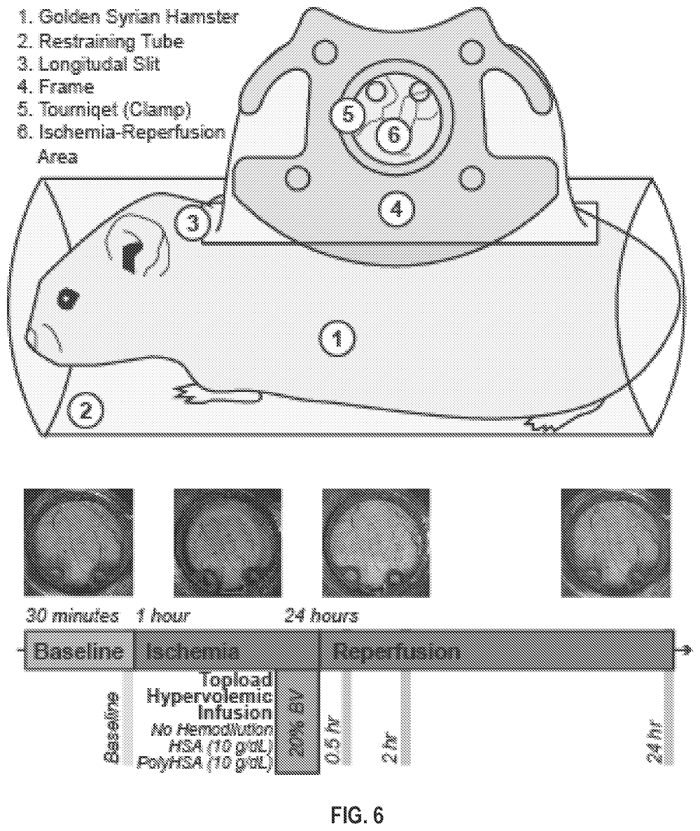

Animals were awake and unanesthetized in a Plexiglass tube during the protocol. The chamber window protruded from a longitudinal slit on the restraining tube. Animals were given 30 minutes to adjust to the environment before baseline measurements of the systemic and microcirculatory hemodynamics were taken. The chamber window was then fixed to the stage of a transillumination intravital microscope (BX51WI, Olympus). The tissue image was projected onto a charge-coupled device camera (COHU 4815) connected to a videocassette recorder (AG-7355; JVC). Measurements were carried out using a 40× (LUMPFL-WIR, numerical aperture 0.8, Olympus) water immersion objective. Ischemia was induced for 1 hour within the tissue in the chamber window via a clamp that compressed a thin, flat rubber ring at the periphery of the window. Perfusion within the chamber window was halted by tightening a precision threaded screw sized to the intact skin side of the chamber window. Microvascular blood flow was continuously monitored to confirm stoppage of blood flow without compression injury. The ischemic period was held for 1 hour after flow was halted within the chamber window. 5 minutes prior to the end of the ischemic period, a post-ischemia topload (hypervolemic) infusion with HSA or PolyHSA equivalent to 20% of the hamster's blood volume (calculated as 7% of the bodyweight) was injected through the jugular vein at 0.1 mL/min. A timeline of the ischemia-reperfusion model and protocol is shown in FIG. 6.

Experimental Groups

24 animals were included in this study. Animals were divided into three experimental groups: no hemodilution (control, no topload infusion given, n=8), infusion of HSA (human serum albumin at 10 g protein per dL fluid, n=8), and infusion PolyHSA (polymerized HSA at 10 g protein per dL fluid, n=8). An additional twelve animals were included as shams that were not subjected to ischemia-reperfusion, but were used to study the extravasation of fluorescent proteins under basal conditions.

Systemic Parameters

MAP and HR were measured continuously at the observation windows (0.5 hours, 2 hours, 24 hours) (MP150, Biopac system).

Microvascular Hemodynamics

Arteriolar and venular blood flow velocities were measured using the photodiode cross-correlation method (Photo-Diode/Velocity, Vista Electronics)²². The measured centerline velocity (V) was corrected according to blood vessel size to obtain the mean RBC velocity²³. A video image shearing method was used to calculate the blood vessel diameter (D). Blood flow (Q) was calculated from the measured values as Q=π·V (D/2)².

Leukocyte-Endothelium Interaction

Adhesion of leukocytes to the endothelium in venules was quantified with low light fluorescent microscopy (ORCA 9247, Hamamatsu) of leukocytes labeled via intravenous injection of acridine orange (5 mg/kg in saline) as previously described^{17,24}. Leukocytes were counted along a 100 μm length segment and categorized as free-flowing, rolling, or immobilized.

Vascular Permeability

Vascular permeability was assessed by measuring the extravasation of Texas-red conjugated dextran (Texas Red-Dextran; 40 kDa MW; Sigma, St. Louis, MO). Animals received a single 100 μL bolus of Texas Red-Dextran (10 mg/mL) in the venous catheter which was used to track vascular permeability throughout the experiment. The dye was allowed to circulate for 5 minutes, and locations of interest (containing arterioles, venules, and tissue) were selected prior to fluorescent imaging. The tissue was excited using a standard Texas Red filter cube and images were recorded using a high light-sensitive camera (C4742-95, Hamamatsu Photonics, Japan). Images of regions of interest were recorded at baseline and 6 h and 24 h after ischemia and reperfusion. A constant camera exposure time was used throughout the experiment. The images were analyzed offline by measuring the relative pixel intensity inside the microvessels (IV) and in the tissue adjacent to the microvessels (EV). Data is displayed as EV/IV, and high ratios indicate increased vascular permeability.

HSA and PolyHSA Extravasation

Extravasation of HSA and PolyHSA was assessed by measuring the fluorescent intensity of FITC-conjugated HSA and PolyHSA. Fluorescein isothiocyanate (Sigma Aldrich) was conjugated to HSA and PolyHSA and dialyzed for 4 and 2 days against saline and distilled water, respectively²⁵. Animals received a single 100 μL bolus of FITC-HSA or FITC-PolyHSA (10 mg/mL) in the venous catheter, which was used to measure the extravasation of the molecule of interest throughout the experiment. Imaging and data processing were performed as described in the Vascular Permeability section, using a standard FITC fluorescence filter cube. Data is displayed as ratio between extravascular and intravascular (EV/IV) fluorescence, where higher ratios indicate increased tissue extravasation of the molecule of interest.

Tissue Apoptosis and Necrosis

Apoptotic and necrotic cells were labeled in situ via infusion of propidium iodide (PI) and Annexin V (0.14 mg each in 140 μL saline per animal; Molecular Probes, Eugene, OR). The dyes circulated for 30 minutes before images were acquired with a high-light sensitive camera (C4742-95, Hamamatsu Photonics). Forty microscopic fields were captured in each animal. Hair follicles and sebaceous glands were excluded from cell counts due to their consistently high rates of necrosis and apoptosis.

Statistics and Reproducibility

Results are presented as mean±standard deviation. All box plots are presented with the median on the centerline; the box limits are set to the upper (75%) and lower (25%) quartile. All outliers are shown in each plot. Data analysis was performed in R (v 4.0.0) using the rstatix (v 0.3.1) package. Data between groups were analyzed with a two-way analysis of Variance (ANOVA) with Tukey's test for posthoc analysis. When possible, parameters were compared against baseline in the same animal or same vessel as a ratio relative to the baseline. For all tests, P<0.05 was considered statistically significant. 6 animals/12 vessels were included in each treatment group.

### REFERENCES

1. M. Nour, M., Scalzo, F. & Liebeskind, D. S. Ischemia-Reperfusion Injury in Stroke. Interv. Neurol. 1, 185-199 (2012).
2. Nelson, D. P. et al. Myocardial immediate early gene activation after cardiopulmonary bypass with cardiac ischemia-reperfusion. Ann. Thorac. Surg. 73, 156-162 (2002).
3. Kosieradzki, M. & Rowinski, W. Ischemia/Reperfusion Injury in Kidney Transplantation: Mechanisms and Prevention. Transplant. Proc. 40, 3279-3288 (2008).
4. Abu-Amara, M. et al. Liver ischemia/reperfusion injury: Processes in inflammatory networks—A review. Liver Transplantation 16, 1016-1032 (2010).
5. Whitson, B. A. et al. Risk factors for primary graft dysfunction after lung transplantation. J. Thorac. Cardiovasc. Surg. 131, 73-80 (2006).
6. PARK, J.-W, BRAUN, P., MERTENS, S. & HEINRICH, K. W. Ischemia: Reperfusion Injury and Restenosis after Coronary Angioplasty. Ann. N. Y. Acad. Sci. 669, 215-236 (1992).
7. Warach, S. & Latour, L. L. Evidence of reperfusion injury, exacerbated by thrombolytic therapy, in human focal brain ischemia using a novel imaging marker of early blood-brain barrier disruption. Stroke 35, 2659-2661 (2004).
8. Kalogeris, T., Baines, C. P., Krenz, M. & Korthuis, R. J. Cell Biology of Ischemia/Repertfusion Injury. in International Review of Cell and Molecular Biology 298, 229-317 (Elsevier Inc., 2012).
9. Yapca, O. E., Borekci, B. & Suleyman, H. Ischemia-reperfusion damage. Eurasian Journal of Medicine 45, 126-127 (2013).
10. Turer, A. T. & Hill, J. A. Pathogenesis of myocardial ischemia-reperfusion injury and rationale for therapy. American Journal of Cardiology 106, 360-368 (2010).
11. Yang, Q., He, G. W., Underwood, M. J. & Yu, C. M. Cellular and molecular mechanisms of endothelial ischemia/reperfusion injury: Perspectives and implications for postischemic myocardial protection. American Journal of Translational Research 8, 765-777 (2016).
12. Douglas, W. W. Stimulus-secretion coupling: variations on the theme of calcium-activated exocytosis involving cellular and extracellular sources of calcium. Ciba Found. Symp. 61-90 (1978). doi:10.1002/9780470720356.ch4
13. Oz, M. C., FitzPatrick, M. F., Zikria, B. A., Pinsky, D. J. & Durán, W. N. Attenuation of microvascular permeability dysfunction in postischemic striated muscle by hydroxyethyl starch. Microvasc. Res. 50, 71-79 (1995).
14. Kauvar, D. S., Baer, D. G., Dubick, M. A. & Walters, T. J. Effect of Fluid Resuscitation on Acute Skeletal Muscle Ischemia-Reperfusion Injury after Hemorrhagic Shock in Rats. J. Am. Coll. Surg 202, 888-896 (2006).
15. Wiedermann, C. J. & Eisendle, K. Comparison of hydroxyethyl starch regulatory summaries from the Food and Drug Administration and the European Medicines Agency. J. Pharm. Policy Pract. 10, (2017).
16. Roberts, I. et al. Hydroxyethyl starch solutions and patient harm. The Lancet 391, 736 (2018).
17. Cabrales, P., Tsai, A. G. & Intaglietta, M. Perfluorocarbon in Microcirculation During Ischemia Reperfusion. J. Am. Coll. Surg. 204, 225-235 (2007).
18. Flaim, S. F. Pharmacokinetics and side effects of perfluorocarbon-based blood substitutes. Artif Cells, Blood Substitutes, Biotechnol. 22, 1043-1054 (1994).
19. Messmer, C., Yalcin, O., Palmer, A. F. & Cabrales, P. Small-volume resuscitation from hemorrhagic shock with polymerized human serum albumin. Am. J. Emerg. Med. 30, 1336-1346 (2012).
20. Wang, G. et al. Shear Stress Regulation of Endothelial Glycocalyx Structure Is Determined by Glucobiosynthesis. Arterioscler. Thromb. Vasc. Biol. 40, 350-364 (2020).
21. Elmer, J., Cabrales, P., Wang, Q., Zhang, N. & Palmer, A. F. Synthesis and biophysical properties of polymerized human serum albumin. Biotechnol. Prog. 27, 290-296 (2011).
22. Intaglietta, M., Silverman, N. R. & Tompkins, W. R. Capillary flow velocity measurements in vivo and in situ by television methods. Microvasc. Res. 10, 165-179 (1975).
23. Lipowsky, H. H. & Zweifach, B. W. Application of the ‘two-slit’ photometric technique to the measurement of microvascular volumetric flow rates. Microvasc. Res. 15, 93-101 (1978).
24. Childs, E. W. et al. In Vivo Visualization of Reactive Oxidants and Leukocyte-Endothelial Adherence Following Hemorrhagic Shock. Shock 18, 423-427 (2002).
25. SCHILLER, A. A., SCHAYER, R. W. & HESS, E. L. Fluorescein-conjugated bovine albumin; physical and biological properties. J. Gen. Physiol. 36, 489-506 (1953).
26. Castro, C., Ortiz, D., Palmer, A. F. & Cabrales, P. Hemodynamics and tissue oxygenation after hemodilution with ultrahigh molecular weight polymerized albumin. Minerva Anestesiol. 80, 537-546 (2014).
27. Varga, R. et al. Effects of colloid solutions on ischemia-reperfusion-induced periosteal microcirculatory and inflammatory reactions: Comparison of dextran, gelatin, and hydroxyethyl starch*. Crit. Care Med. 36, 2828-2837 (2008).
28. Steinbauer, M., Harris, A. G., Leiderer, R., Abels, C. & Messmer, K. Impact of dextran on microvascular disturbances and tissue injury following ischemia/reperfusion in striated muscle. Shock 9, 345-351 (1998).
29. Steinbauer, M., Harris, A. G. & Messmer, K. Effects of dextran on microvascular ischemia-reperfusion injury in striated muscle. Am. J. Physiol. —Hear. Circ. Physiol. 272, (1997).
30. Sigurjonsson, J., Hedman, D., Bansch, P. & Schött, U. Comparison of dextran and albumin on blood coagulation in patients undergoing major gynaecological surgery. Perioper. Med. 7, 21 (2018).
31. Rehm, M. et al. Shedding of the endothelial glycocalyx in patients undergoing major vascular surgery with global and regional ischemia. Circulation 116, 1896-1906 (2007).
32. Reitsma, S., Slaaf, D. W., Vink, H., Van Zandvoort, M. A. M. J & Oude Egbrink, M. G. A. The endothelial glycocalyx: Composition, functions, and visualization. Pflugers Archiv European Journal of Physiology 454, 345-359 (2007).
33. Connors, J. M. & Levy, J. H. COVID-19 and its implications for thrombosis and anticoagulation. Blood 135, 2033-2040 (2020).
34. Lazarian, G. et al. Autoimmune haemolytic anaemia associated with COVID-19 infection. Br. J. Haematol. 190, 29-31 (2020).

### Example 2: Polymerized Albumin Restores Impaired Hemodynamics in Endotoxemia and Polymicrobial Sepsis

Abstract

Background: Fluid resuscitation following severe inflammation-induced hypoperfusion is critical for the restoration of hemodynamics and the prevention of multiorgan dysfunction syndrome during septic shock. Fluid resuscitation with commercially available crystalloid and colloid solutions only provides transient benefits, followed by fluid extravasation and tissue edema through the inflamed endothelium. The increased molecular weight (M.W.) of polymerized human serum albumin (PolyHSA) can limit fluid extravasation, leading to restoration of hemodynamics.

Methods: In this prospective study, we evaluated how fluid resuscitation with PolyHSA impacts the hemodynamic and immune response in a lipopolysaccharide (LPS) induced endotoxemia mouse model. Additionally, we evaluated fluid resuscitation with PolyHSA in a model of polymicrobial sepsis induced by cecal ligation and puncture (CLP).

Results: Resuscitation with PolyHSA attenuated the immune response as demonstrated by decreased pro-inflammatory cytokines and the number of leukocytes adhered to the endothelium. Additionally, resuscitation with PolyHSA improved the maintenance of systemic hemodynamics and restoration of microcirculatory hemodynamics. This decrease in inflammatory immune response and maintenance of vascular wall shear stress likely contributes to the maintenance of vascular integrity following fluid resuscitation with PolyHSA.

Conclusions: The sustained restoration of perfusion, decrease in pro-inflammatory immune response, and improved vascular integrity that results from the high MW of PolyHSA indicates that a PolyHSA based solution is a potential resuscitation fluid for endotoxic and septic shock.

Background

Sepsis associated mortality and morbidity stem from host-pathogen interactions that can continue long after the initial insult is treated(1). These interactions lead to systemic inflammatory response syndrome (SIRS), which can result in multiorgan dysfunction syndrome (MODS) if not effectively controlled(2,3). Sepsis is the most common cause of non-coronary deaths in intensive care units, and the care and treatment of sepsis costs approximately $20 billion annually in the United States(4,5). Proper treatment of sepsis and septic shock has been a controversial research topic due to conflicting results between different pre-clinical models and between different clinical observations(6). These likely stem from the different etiologies of conditions and infectious agents that precede the insult of sepsis. In addition to controlling and eliminating the initial insult, vasopressor therapies, fluid resuscitation strategies, and the combination of the two, are the most common treatments of sepsis.

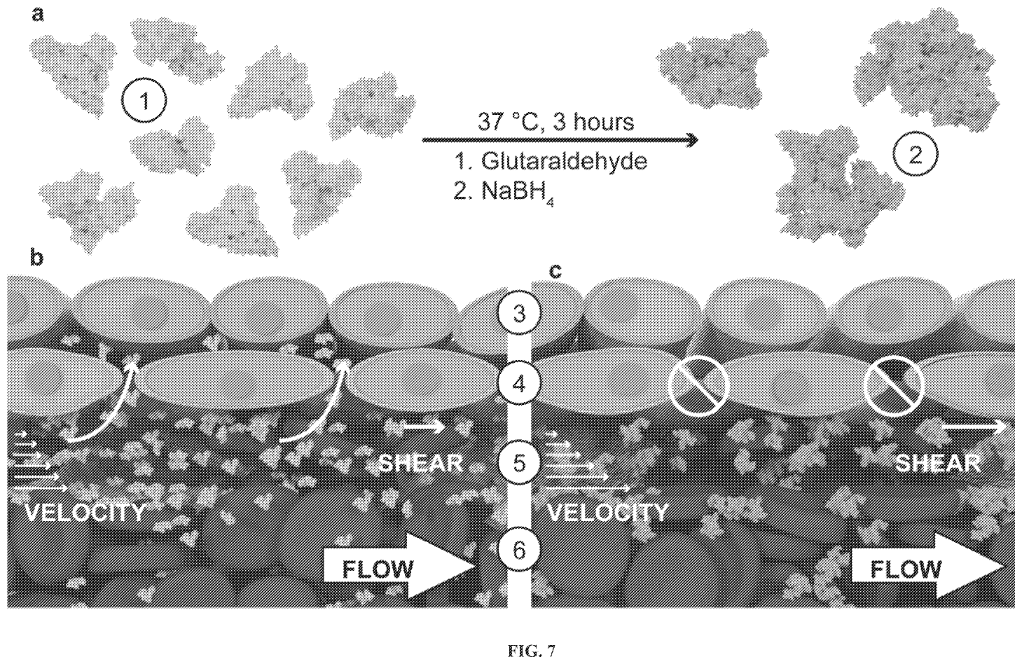

The goal of vasopressor therapy is to maintain blood pressure and flow to vital organs by restricting blood flow to other tissues, such as the skin and gut. Both treatment strategies have been heavily criticized. Some investigators have found that vasopressor therapy can result in impaired gut and sublingual microcirculatory blood flow(7,8), while other investigators found no such detriments to the microcirculation(9). However, restoration of hemodynamic stability via fluid resuscitation is vital to alleviate sepsis-induced hypoperfusion that can result in multiorgan failure(10,11). Fluid resuscitation strategies using crystalloids or colloids have been criticized, as they typically only show transient patient benefits, followed by edema and acute respiratory failure. After the initial resuscitation has been completed, septic shock is in part pathophysiologically characterized by deterioration of the vascular endothelial barrier(12). As a result, small colloidal proteins, such as human serum albumin (HSA), can extravasate from the vascular space into the tissue space, decreasing intravascular, and increasing extravascular colloidal osmotic pressure (COP), which promotes ultrafiltration and prevents reabsorption of fluid (FIG. 7b). As such, a fluid resuscitation therapy that can maintain intravascular oncotic pressure is necessary for the proper care of septic patients.

Hydroxyethylene starch (HES) solutions have been routinely used for fluid resuscitation from septic shock. HES solutions resolve the loss in intravascular COP due to their larger molecular size compared to native colloidal proteins. However, the U.S. Food and Drug Administration recently vetoed the use of HES solutions due to serious adverse effects such as unexpected coagulopathies and renal injury(13,14). Previously, we have used polyethylene glycol (PEG) surface conjugated bovine serum albumin (BSA) solutions to improve recovery of functional capillary density (FCD) and tissue oxygenation following lipopolysaccharide (LPS) induced endotoxemia in hamsters(15). PEGylation significantly increases the molecular size of the BSA molecules, and the hydrophilic nature of PEG increases the oncotic pressure that the molecule can apply. However, the PEGylation process is costly and only increases the COP without increasing the solution viscosity. The cost of PEGylated proteins precludes their use in the generation of plasma expanders from widespread commercialization, and the low solution viscosity of PEG-BSA decreases blood viscosity reducing endothelial shear stress. Alternatively, glutaraldehyde-based protein polymerization is a simple, cost-effective, and scalable strategy to increase the molecular diameter and oncotic pressure of HSA solutions(16-18). Glutaraldehyde non-specifically reacts with surface proteins to form inter and intramolecular crosslinks between HSA molecules, which can result in significant increases in the effective molecular diameter. A schematic of this process is shown in FIG. 7A. Unlike unmodified HSA, the increased size of glutaraldehyde polymerized HSA (PolyHSA) restricts extravasation and increases solution viscosity, which restores blood viscosity. The increase in intravascular retention, combined with a functional reduction in extravascular COP and an increase in plasma viscosity, restores endothelial shear stress, which may lead to improved maintenance of macro and micro-hemodynamics(17).

Based on PolyHSA's ability to maintain blood volume, restore blood viscosity, and hemodynamics, we hypothesize that fluid resuscitation with a PolyHSA solution would improve the maintenance of macro- and micro-circulatory hemodynamics during a controlled septic shock model with systemic inflammation induced by LPS infusion. Endotoxemia was induced via administration of LPS to reduce microvascular blood flow and FCD. Intravital microscopy was used to assess how fluid resuscitation with PolyHSA influences in hemodynamics (vascular tone, blood flow, and FCD). In addition, mean arterial blood pressure (MAP) and heart rate were used as indicators of systemic circulatory response. To assess the impact of fluid therapy on the tissue, endothelial permeability, and cell death (apoptosis and necrosis) were studied in situ in the same tissue where microvascular hemodynamics were observed. Changes in immune response were assessed by monitoring leukocyte interaction in the vasculature and serum cytokine levels. In addition to the LPS model, we also investigated the effects of fluid resuscitation with PolyHSA in a hamster model of polymicrobial sepsis induced by cecal ligation and puncture (CLP).

Methods

Polymerized Human Serum Albumin Synthesis

The HSA (Albuminar®) used in this study was obtained from ABO Pharmaceuticals, San Diego, CA. Polymerization of HSA was performed as previously described(18). In brief, HSA was incubated with glutaraldehyde at a 30:1 molar ratio of glutaraldehyde to HSA. The polymerization reaction was incubated for 3 hours at 37° C. The reaction was quenched with sodium borohydride (NaBH₄). After the reaction, the resulting PolyHSA was diafiltered into a modified Ringer's Lactate solution (115 mM NaCl, 4 mM KCl, 1.4 mM CaCl₂, 13 mM NaOH, 27 mM sodium lactate, and 2 g/L N-acetyl-L-cysteine) on a 100 kDa polysulfone hollow fiber filter (Spectrum Labs, Rancho Dominguez, CA) for four diafiltration cycles. All PolyHSA samples were filtered through a 0.2 μm filter. The viscosity of unmodified HSA and PolyHSA solutions was measured with a DV-II+ cone and plate viscometer (Brookfield Engineering Laboratories, Middleboro, MA) at a shear rate of 160 s⁻¹ (17,18). The COP of the solutions were measured with a 4420 membrane colloid osmometer (Wescor, Logan, UT).

Animal Preparation

All the procedures, including animal handling and care, followed the National Institutes of Health (NIH) Guide for the Care and Use of Laboratory Animals. The U.C. San Diego Institutional Animal Care and Use Committee approved the experimental protocol. Mice and hamsters were fitted with a dorsal skinfold window chamber for direct visualization of the microcirculation. This model has been used widely to characterize the perfusion of peripheral tissues in unanesthetized animals, as previously described(19). Briefly, animals were anesthetized with sodium pentobarbital (50 mg/kg i.p.), the dorsal area depilated, and the skinfold was lifted from the back using sutures. The skinfold was then captured between two titanium frames, each with a circular opening for visualization. The skin on one side of the window chamber was removed, following the outline of the circular window, and the exposed skin was covered with a glass coverslip. Two days after window chamber implantation, the mice and hamsters were anesthetized again, and a heparinized catheter was implanted in the left common carotid artery. Mice were then allowed 2 additional days for recovery before any experimental procedures were performed, and hamsters were immediately subjected to the CLP procedure, as described below.

Endotoxemia Protocol

Male Balb/c mice (23-28 g, Jackson Laboratory) were used for this experimental study. All animals were housed under the same conditions until the day of the experiment (12 hr day/night cycles; approximately 25° C. and 60% humidity). Only animals within the defined inclusion criteria were used in this study. Baseline parameters were collected after acclimatizing to the experimental environment for at least 15 minutes. Animals received 10 mg/kg of lipopolysaccharide (LPS) from E. coli serotype 0128:B12 (Sigma, St. Louis, MO), suspended in 0.1 mL of saline via the arterial catheter. Fluid resuscitation was performed 1 hour after LPS injection in the relevant groups and consisted of a single infusion of 30% of the animal's blood volume (estimated as 7% of body weight) over 10 minutes. No additional therapies were given. Food and water were available ad libitum between observation time points. All animals survived the experimental protocol.

Cecal Ligation and Puncture Protocol

Male Golden Syrian Hamsters (50-70 g, Charles Rivers Laboratories) were used for this study. A state of polymicrobial sepsis was induced using the CLP model as described elsewhere(20,21). Briefly, animals were as described above. After shaving and disinfection of the animal's abdomen, a 1 to 2 cm laparotomy was performed in the left flank and the cecum was exteriorized. Then, the cecum was ligated using a sterile silk suture and perforated near the base 2 times using a 20-gauge needle. Before replacing the cecum in the abdominal cavity, it was gently pressed to release part of the intestinal content. The laparotomy was closed and the animal was left to recover in a 37° C. heating pad. To avoid excessive surgical interventions in a single animal, we performed the CLP procedure during the same surgical session used for catheter implantation. Fluid resuscitation was performed 1 hour after LPS injection in the relevant groups and consisted of a single infusion of 30% of the animal's blood volume (estimated as 7% of body weight) over 10 minutes. No additional therapies were given. All animals survived the initial CLP procedure. Microvascular and systemic monitoring of the animals began 1 hour after full recovery from anesthesia. Continued survival was monitored for 4 days following the initial procedure.

Inclusion Criteria

Animals were considered suitable for the experiments if: 1) systemic parameters were within normal range at baseline, namely, heart rate (HR)>350 beats/min and mean arterial pressure (MAP)>100 mmHg; and 2) microscopic examination of the tissue in the window chamber did not reveal signs of edema or bleeding under 650× magnification.

Experimental Groups

Eighteen (18) animals were included in each arm (endotoxemia and polymicrobial sepsis) of this study and were divided into three experimental groups, named by the resuscitation fluid given (or lack thereof), namely: No resuscitation (no fluid resuscitation given, n=6); HSA (human serum albumin at 10 g protein per dL fluid, n=6); and PolyHSA (polymerized human serum albumin at 10 g protein per dL fluid, synthesized as described above, n=6).

Systemic Parameters

MAP and HR were monitored continuously during the observation periods using the arterial line and a transducer-computer interface (MP150; Biopac Systems, Santa Barbara, CA).

Microcirculatory Hemodynamics

The window chamber was studied using transillumination on an upright microscope (BX51WI, Olympus, New Hyde Park, NY). Measurements were carried out using a 40× water immersion objective (LUMPFL-WIR, numerical aperture 0.8, Olympus). The microscope was equipped with a high-speed video camera (Fastcam 1024 PCI, Photron, USA), which was used to record videos of the microcirculation at 1000 frames per second. Briefly, the animals were restrained in a plexiglass tube with a longitudinal opening from which the window chamber protruded. Animals were then fixed to the stage of the microscope. Individual arterioles were identified at baseline based on visual clarity and followed throughout the experiment to improve statistical power. At each time point, a video recording of the individual vessels was captured and then analyzed offline as previously described(22). The volumetric flow rate was estimated from the measured diameter (D) and centerline velocity (V) as Q=π·V (D/2)². Shear stress was estimated from the measured values as τ=8V/D.

Functional Capillary Density

Functional capillary density (FCD) was measured by counting the number of capillaries with a transit of at least a single red blood cell in a 45 s period. Ten consecutive microscopic visual fields are selected at baseline and monitored at different time points throughout the experiment.

Tissue Apoptosis and Necrosis

Apoptotic and necrotic cells are labeled in situ by infusion of propidium iodide (P.I.) and Annexin V (0.14 mg each in 140 μL saline per animal; Molecular Probes, Eugene, OR). The dye was allowed to circulate for 30 minutes. Images were acquired using a high light-sensitive camera (C4742-95, Hamamatsu Photonics, Japan). A total of 40 microscopic fields were captured per animal, and the number of single-labeled and double-labeled cells were counted at each time point. Hair follicles and sebaceous glands were excluded from cell counts due to their consistently high rates of necrosis and apoptosis.

Endothelial Barrier Permeability

Microvascular wall permeability was assessed by measuring the extravasation rate of fluorescein isothiocyanate conjugated dextran (FITC-Dextran; 70 kDa M.W.; Sigma, St. Louis, MO). The animals received a 100 μL bolus of FITC-Dextran (10 mg/mL) in the tail vein. The dye was allowed to circulate for 5 minutes, and locations of interest (containing arterioles, venules, and tissue) were selected prior to fluorescent imaging. The tissue was excited using a standard FITC filter cube, and images were recorded using a high light-sensitive camera (C4742-95, Hamamatsu Photonics, Japan). Images of the regions of interest were recorded at baseline and 6 h after LPS induction. A constant camera exposure time was used throughout the experiment. The images were analyzed offline by measuring the relative pixel intensity inside the microvessels (IV) and in the tissue adjacent to the microvessels (E.V.). Data was displayed as EV/IV, and high ratios indicate increased vascular permeability.

Leukocyte Endothelial Interaction

To label leukocytes, animals received a 100 μL bolus of Rhodamine 6G (5 mg/kg; Sigma, St. Louis, MO) 5 minutes before the time point of interest. Fluorescently labeled leukocytes were excited, and images were captured with a Vivid Set (XF104-2 filter, Omega Filters, Brattleboro, VT) using a high-light sensitive camera (C4742-95, Hamamatsu Photonics, Japan). 60 seconds of video was captured on a straight portion of the vessel at 10 frames per second. During playback, the vessels were segmented into 100 μm lengths, and leukocytes were counted and classified as “rolling” or “adhered” to the endothelium as previously described(23).

Cytokine ELISA Measurements

Plasma samples collected from animals at multiple time points were analyzed using a Multiplex Mice Cytokine ELISA Kit (R&D Systems, Minneapolis, MN) following the manufacturer's instructions.

Statistical Analysis

Results are presented as mean±standard deviation. All box plots are presented with the median on the centerline. The box limits are set to the upper (75%) and lower (25%) quartile. All outliers are shown in each plot. For all tests, P<0.05 was considered statistically significant. Data analysis was performed in R (v 4.0.0) using the rstatix (v 0.3.1). Package. Data between groups were analyzed with a two-way Anova with Tukey's test for post-hoc analysis. When possible, parameters were compared against baseline in the same animal or same vessel as a ratio relative to the baseline. For all tests, P<0.05 was considered statistically significant. Survival data was analyzed with the survival (v 3.2.3) and survminer (v 0.4.7) packages.

Results

Biophysical Properties of PolyHSA

Both formulations were corrected to a total protein concentration of 10 g/dL before transfusion. Polymerization of HSA resulted in decreased COP (18 mm Hg) compared to unmodified HSA (42 mm Hg). Polymerization also resulted in an increase in molecular weight 1 U (410 kDa) compared to unmodified HSA (67 kDa) There were no significant changes in viscosity between the PolyHSA (4.2 cP) and unmodified HSA (1.5 cP).

Endotoxemia Systemic Hemodynamics

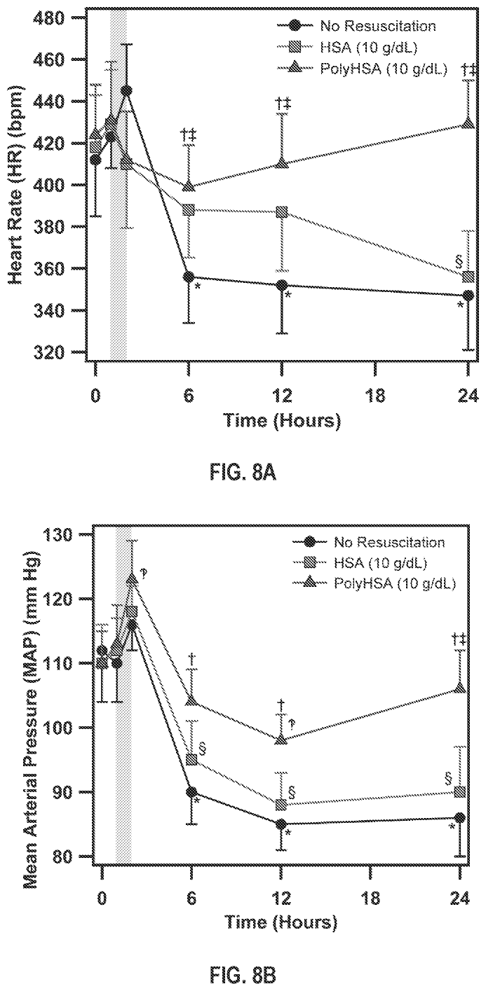

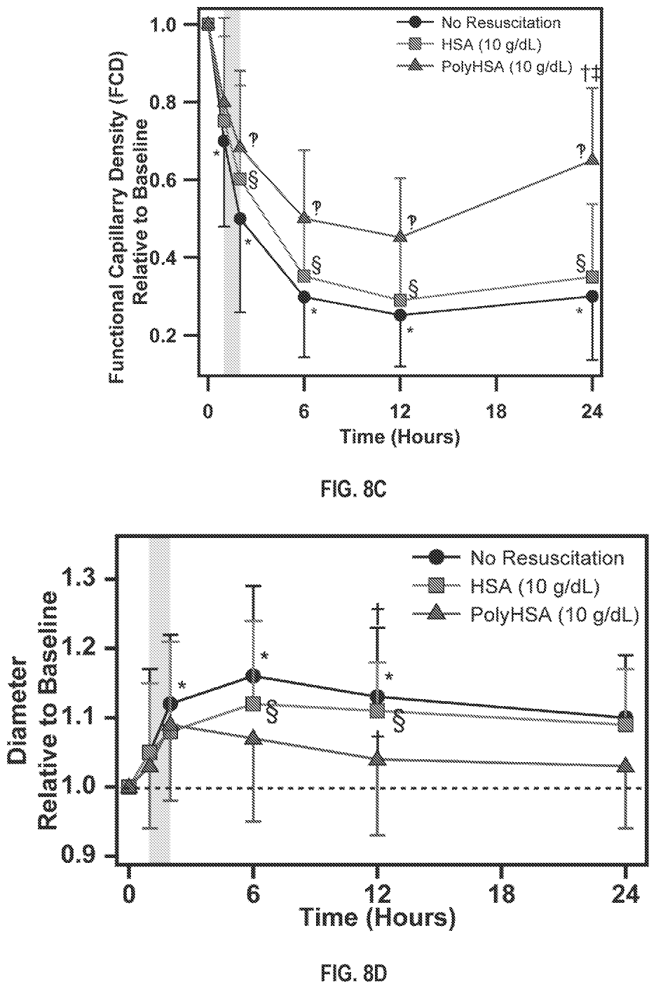

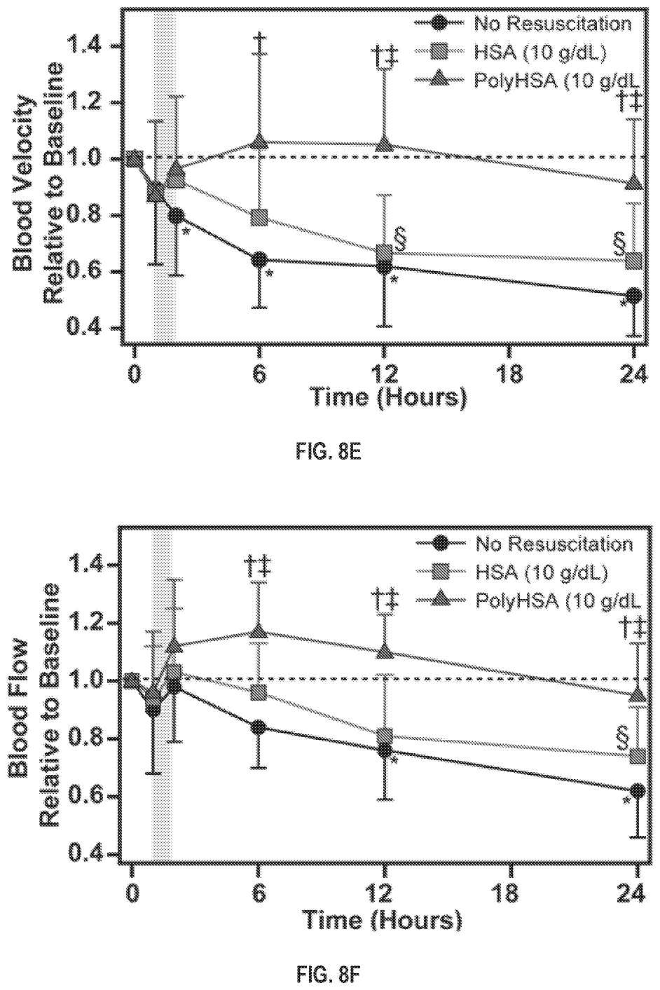

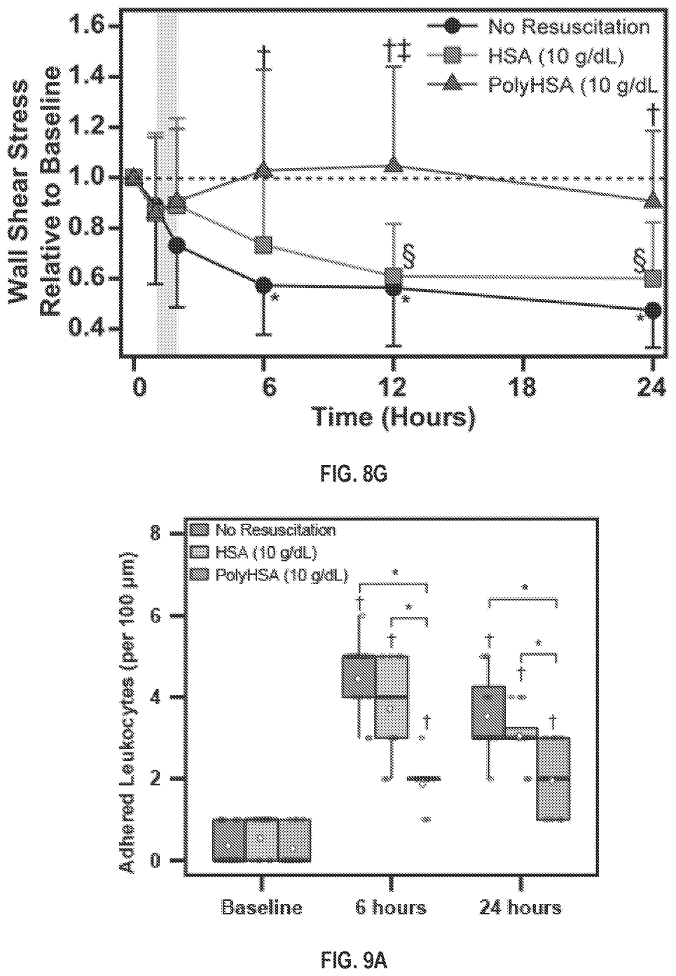

The changes in HR and MAP during LPS induced endotoxemia are depicted in FIG. 8A-8B. All groups had similar HR and MAP at baseline and 1 hour following LPS induced endotoxemia. At 2 hours after administration of LPS, the MAP was significantly greater in animals resuscitated with PolyHSA compared to baseline conditions. Animals that received no resuscitation had significantly (P<0.05) decreased HR and MAP compared to baseline conditions 6 hours after administration of LPS, which persisted throughout the remainder of the protocol. For animals resuscitated with HSA, there was a significant (P<0.05) decrease in MAP 6 hours after administration of LPS, and MAP did not recover. Additionally, after 24 hours, the HR in animals resuscitated with HSA was significantly lower than baseline conditions. There were no other significant differences in HR and MAP compared to the baseline conditions in animals resuscitated with PolyHSA for the entirety of the protocol. From 6 hours to the end of the protocol, animals resuscitated with PolyHSA had significantly (P<0.05) greater MAP compared to animals that received HSA and those that had no fluid resuscitation. At these times, the HR of animals that received PolyHSA was significantly greater than animals that received no fluid resuscitation. Additionally, after 24 hours, animals that received PolyHSA had significantly (P<0.05) greater HR compared to animals resuscitated with HSA.

Endotoxemia Microcirculatory Hemodynamics

Changes in the microcirculatory hemodynamics are shown in FIG. 8D-G. Immediately following LPS induced endotoxemia, the arteriole diameter increased. At the 6 and 12-hour mark, animals that did not receive fluid resuscitation and animals the received HSA had significantly (P<0.05) greater arteriole diameter compared to baseline. Without fluid resuscitation, there was a significant (P<0.05) decrease in arteriole blood velocity and wall shear stress starting 6 hours after LPS administration. In animals that received HSA as the resuscitation fluid, there was a significant (P<0.05) decrease in blood velocity and wall shear stress compared to baseline conditions 12 hours after LPS administration. In these animals, this resulted in significantly (P<0.05) reduced blood flow at 24 hours after LPS administration. For animals resuscitated with PolyHSA, there were no significant changes in arteriole blood velocity and blood flow relative to baseline. Starting 6 hours post LPS administration, animals resuscitated with PolyHSA had significantly (P<0.05) higher arteriole blood velocity and arterial blood flow compared to animals that did not receive fluid resuscitation. Compared to animals that received HSA, the arteriole blood velocity and flow were significantly (P<0.05) greater in animals resuscitated with PolyHSA at 12 hours post LPS administration.

Endotoxemia Functional Capillary Density

Changes in the FCD are shown in FIG. 8C. In all animals, administration of LPS resulted in significantly decreased FCD compared to baseline conditions. The FCD significantly (P<0.05) decreased starting 1 hour after LPS administration for the no resuscitation treatment groups. Starting 2 hours after LPS administration the FCD was significantly lower than baseline conditions in all treatment groups. At the end of 24 hours, the FCD in the HSA and no resuscitation treatment groups was still dramatically decreased to ˜32.5% of the FCD at baseline. In contrast, the FCD was more preserved in animals resuscitated with PolyHSA compared to untreated animals and animals resuscitated with HSA at 6 and 12 hours after LPS administration. The FCD significantly (P<0.05) decreased in the PolyHSA group compared to baseline, and by 24 hours the FCD in animals treated with PolyHSA was significantly (P<0.05) greater than animals in the HSA and no resuscitation treatment groups.

Endotoxemia Leukocyte Endothelial Interaction

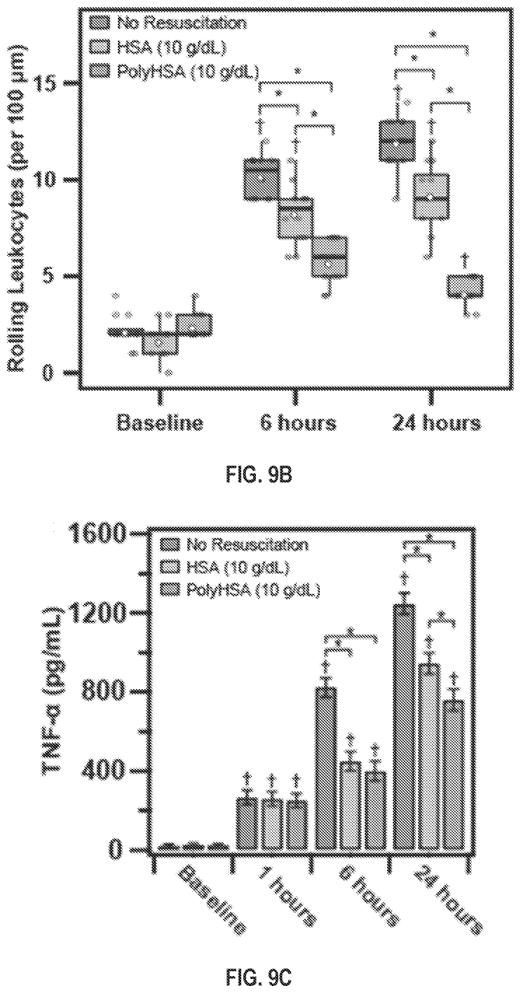

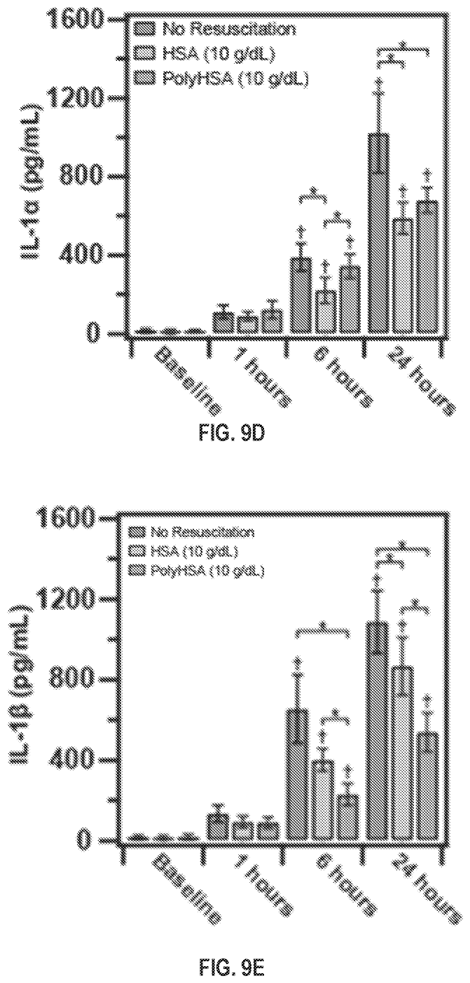

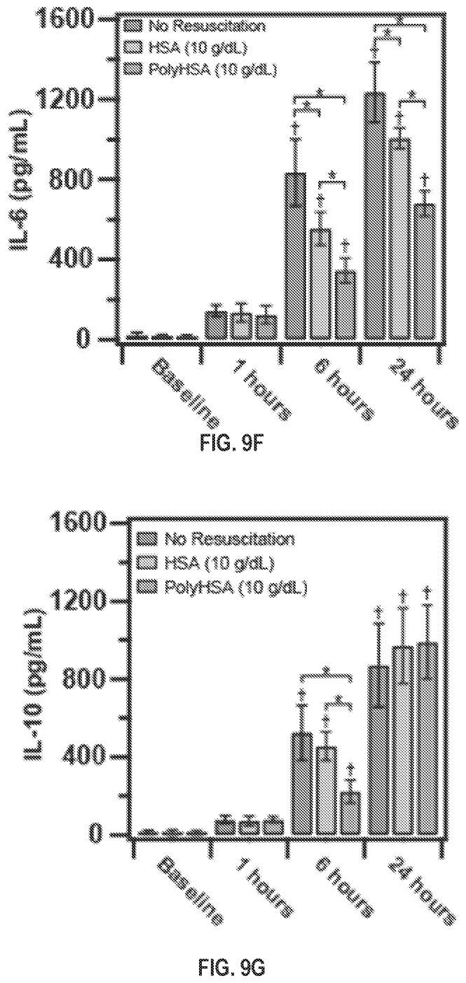

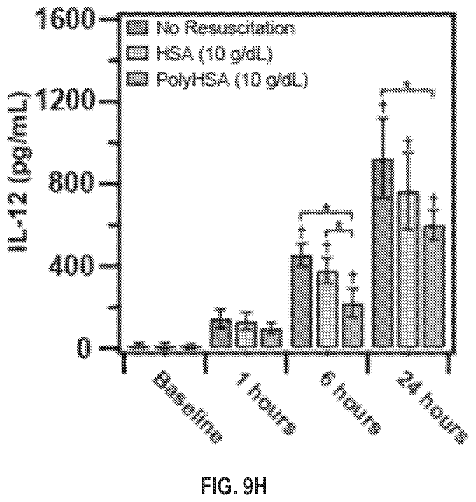

Changes in the number of leukocytes adhered to and rolling on the endothelium is depicted in FIG. 9A-9B. At baseline conditions, there were no significant differences in the number of adhered and rolling leukocytes between each treatment group. For all treatment groups, there was a significant (P<0.05) increase in both adhered and rolling leukocytes after LPS administration. 24 hours after resuscitation with HSA, there were significantly fewer adhered and rolling leukocytes compared to animals that did not receive any fluid resuscitation. In animals resuscitated with PolyHSA, the number of both adhered and rolling leukocytes was significantly (P<0.05) lower compared to the other treatment groups.

Endotoxemia Cytokine ELISA Measurements

Changes in serum cytokines tumor necrosis factor-alpha (TNF-α), interleukin 1-alpha (IL-1α), interleukin 1-beta (IL-1β), interleukin 6 (IL-6), interleukin 10 (IL-10), and interleukin 12 (IL-12) as measured with ELISA are shown in FIG. 9C-9H. At baseline and immediately prior to fluid resuscitation (1 hour after LPS induced endotoxemia), there were no significant differences in cytokines between treatment groups. 1 hour after LPS induced endotoxemia, there were significant (P<0.05) continued increases in serum TNF-α in all treatment groups compared to baseline TNF-α concentration. For all other measured cytokines, there were significant (P<0.05) increases in cytokine levels at 6 hours after LPS induced endotoxemia in each treatment group. For animals resuscitated with HSA and PolyHSA, serum TNF-α and IL-1p levels were significantly lower at 6 and 24 hours after LPS induced endotoxemia compared to animals that received no fluid resuscitation. For animals resuscitated with PolyHSA, serum TNF-α and IL-1β levels were significantly lower compared to animals resuscitated with HSA 24 hours after LPS induced endotoxemia. 6 hours after LPS induced endotoxemia, serum IL-6 levels were significantly lower in animals resuscitated with PolyHSA and HSA compared to animals that underwent no fluid resuscitation. In addition, animals resuscitated with PolyHSA had significantly lower serum IL-6 levels compared to animals treated with HSA 6 hours after LPS induced endotoxemia. At 6 hours, animals resuscitated with HSA had significantly (P<0.05) lower levels of serum IL-1α compared to animals that received no fluid resuscitation. At 24 hours, the levels of serum IL-1α was significantly (P<0.05) lower in both the HSA and PolyHSA treatment groups than the group that received no fluid resuscitation.

6 hours after LPS induced endotoxemia, animals that underwent fluid resuscitation with PolyHSA had significantly (P<0.05) lower levels of serum IL-10 compared to the HSA and no resuscitation treatment groups. However, after 24 hours, there was no significant difference in levels of serum IL-10 between each treatment group. Animals resuscitated with PolyHSA had significantly (P<0.05) lower serum IL-12 levels compared to animals that did not undergo fluid resuscitation at 6 and 24 hours after LPS induced endotoxemia.

Endotoxemia Apoptosis and Necrosis

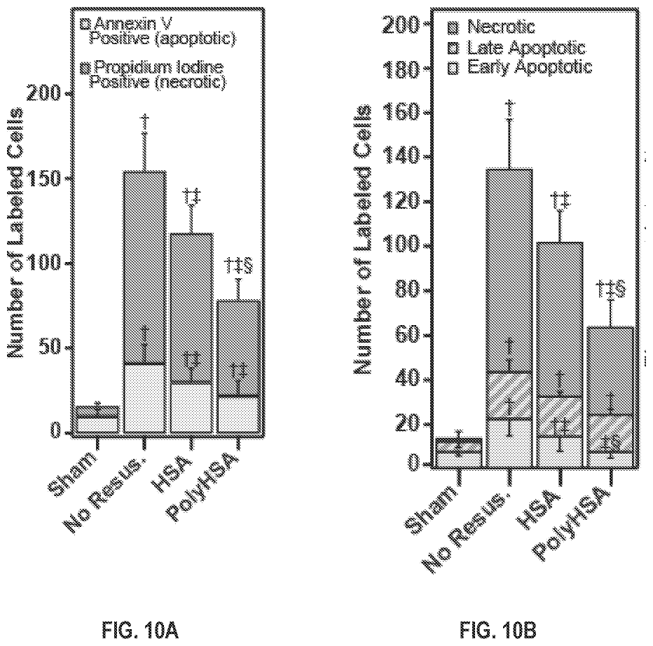

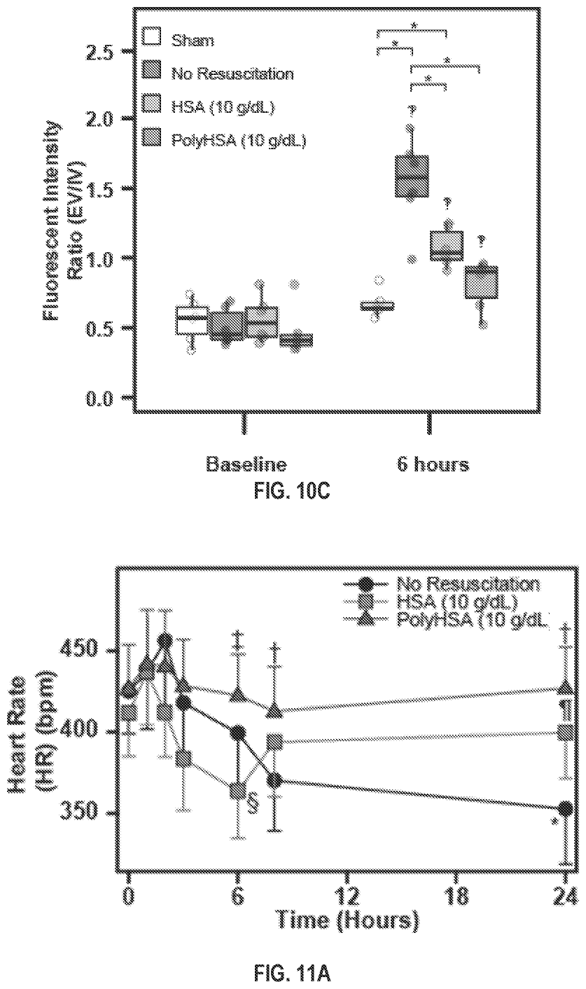

The number of cells labeled with Annexin V and propidium iodide (P.I.) and the corresponding count of necrotic (annexin V+/PI−), early apoptotic (annexin V−/PI+), and late apoptotic (annexin V+/PI+) cells 24 hours after LPS administration is shown in FIG. 10A-10B. For each treatment group, there was a significant (P<0.05) increase in the number of necrotic cells compared to the Sham control. Tissue from animals that were resuscitated with HSA or PolyHSA had significantly (P<0.05) fewer necrotic cells than animals that did not receive fluid resuscitation. Additionally, animals resuscitated with PolyHSA had significantly (P<0.05) fewer necrotic cells than those that received unmodified HSA. Tissue from animals that either received no fluid resuscitation or were treated with HSA had significant (P<0.05) increases in the number of early apoptotic cells. Whereas tissue from animals resuscitated with PolyHSA had no significant difference in early apoptotic cells compared to the control. Each treatment group had a significant increase in the number of late apoptotic cells compared to the control. However, there were no significant differences between the number of late apoptotic cells between each treatment group.

Endotoxemia Endothelial Barrier Permeability

The changes in endothelial permeability measured via extravascular/intravascular (EV/IV) fluorescent signals from FITC-Dextran (70 kDa M.W.) extravasation are shown in FIG. 10C. At baseline conditions, there was no significant difference in the intensity ratio between each treatment group. At 6 hours after LPS, there was a significant (P<0.05) increase in the EV/IV intensity ratio compared to baseline conditions for all treatment groups. Animals that underwent no fluid resuscitation or were resuscitated with HSA had significantly greater EV/IV intensity ratios compared to the Sham control. Resuscitation with HSA and PolyHSA resulted in significantly (P<0.05) decreased EV/IV intensity ratios compared to animals that received no fluid resuscitation. There was no significant difference in EV/IV intensity ratios between the Sham control and animals resuscitated with PolyHSA.

CLP Systemic Hemodynamics

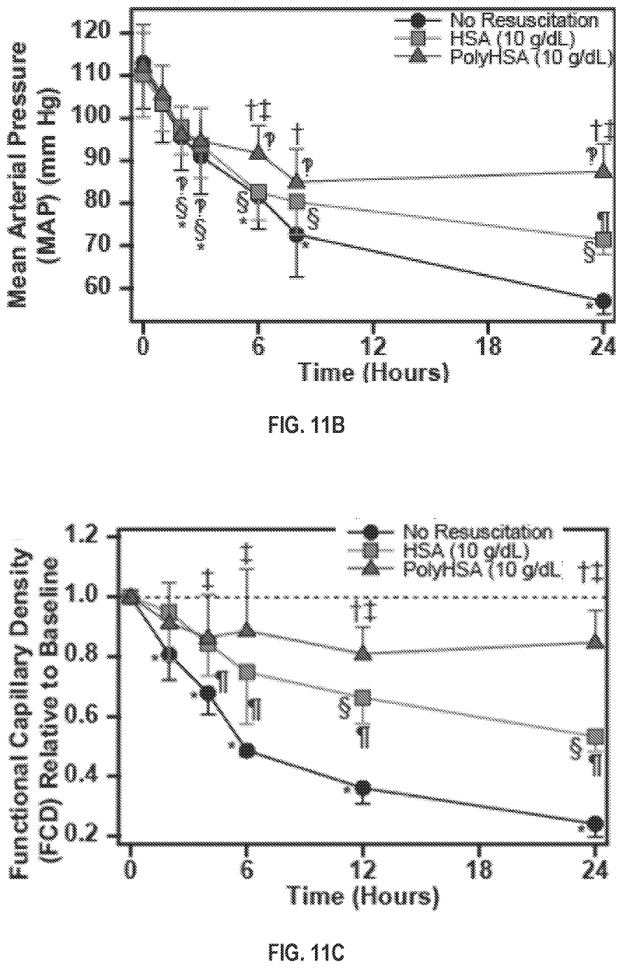

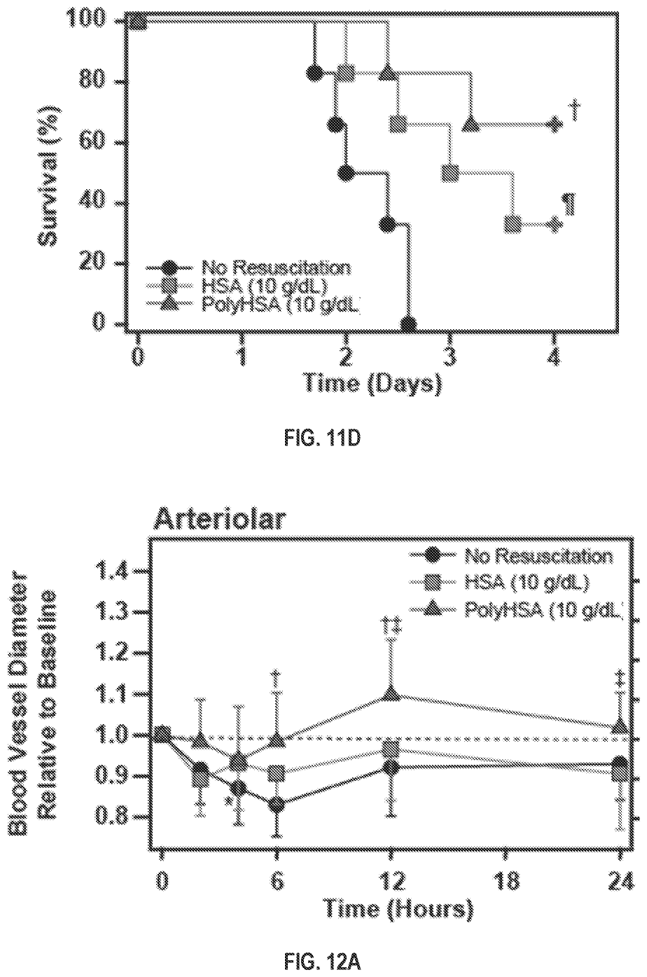

Changes in the HR and MAP in animals that underwent the CLP induced septic shock are shown in FIG. 11A-11B. At 6 hours following the CLP procedure, the HR in the HSA treatment group was significantly lower than baseline conditions and animals in the PolyHSA treatment group at the same time point. At 24 hours, the HR of animals in the HSA treatment group was significantly higher than animals in the no resuscitation group. After 8 hours, animals that received PolyHSA had significantly higher HR than animals that received no fluid resuscitation. Starting 2 hours after the CLP procedure, there was a significant (P<0.05) sustained decrease in MAP. After 6 hours, animals in the PolyHSA treatment group had significantly (P<0.05) higher MAP compared to the no resuscitation group. At 6 and 24 hours, animals in the HSA treatment group had significantly lower MAP compared to animals in the Poly HSA treatment group.

CLP Microcirculatory Hemodynamics

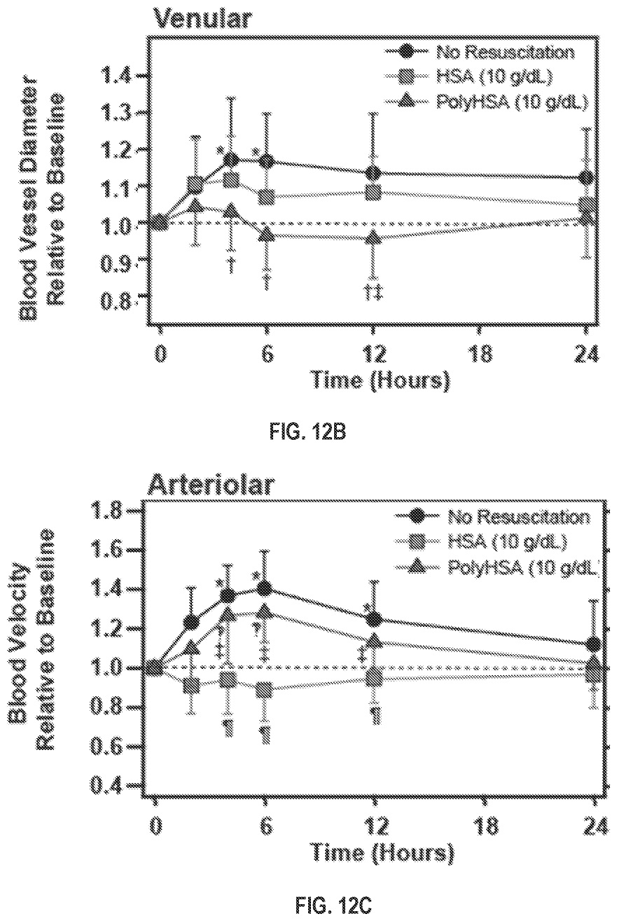

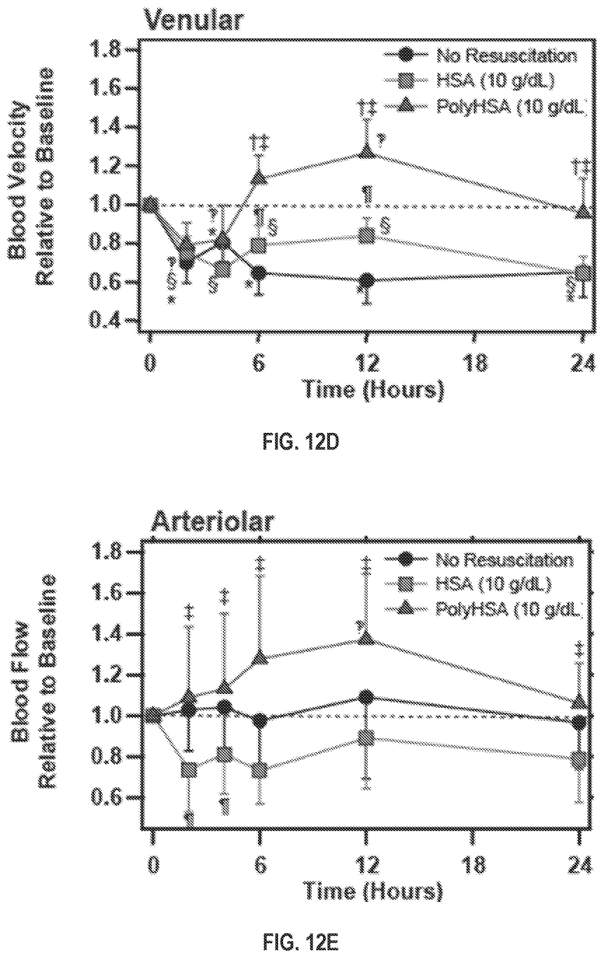

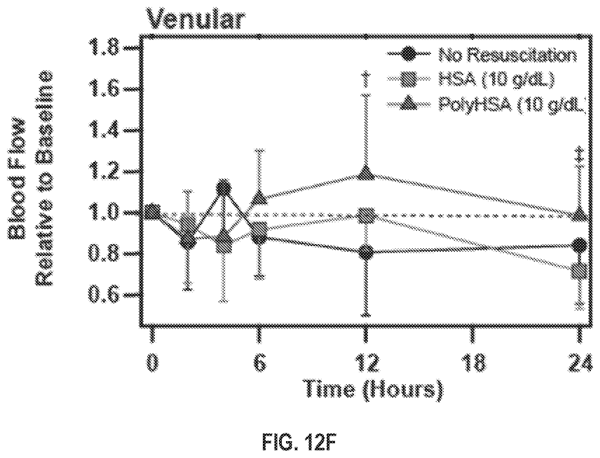

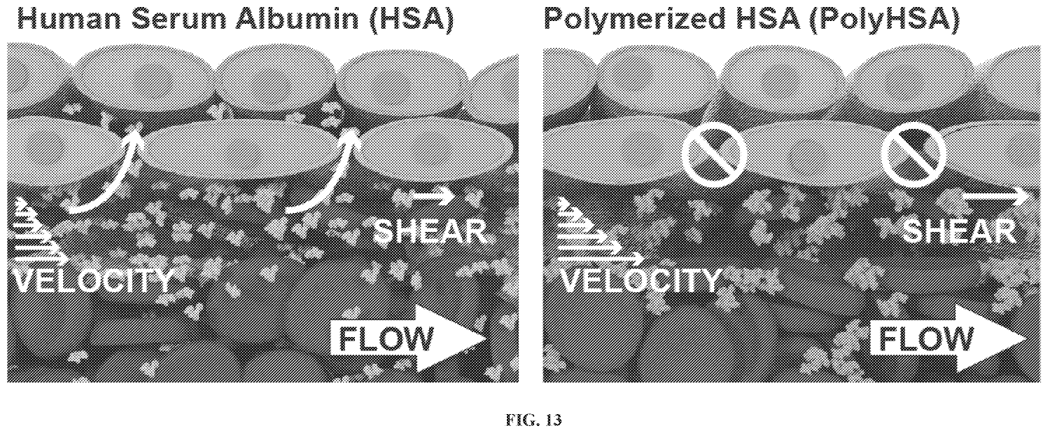

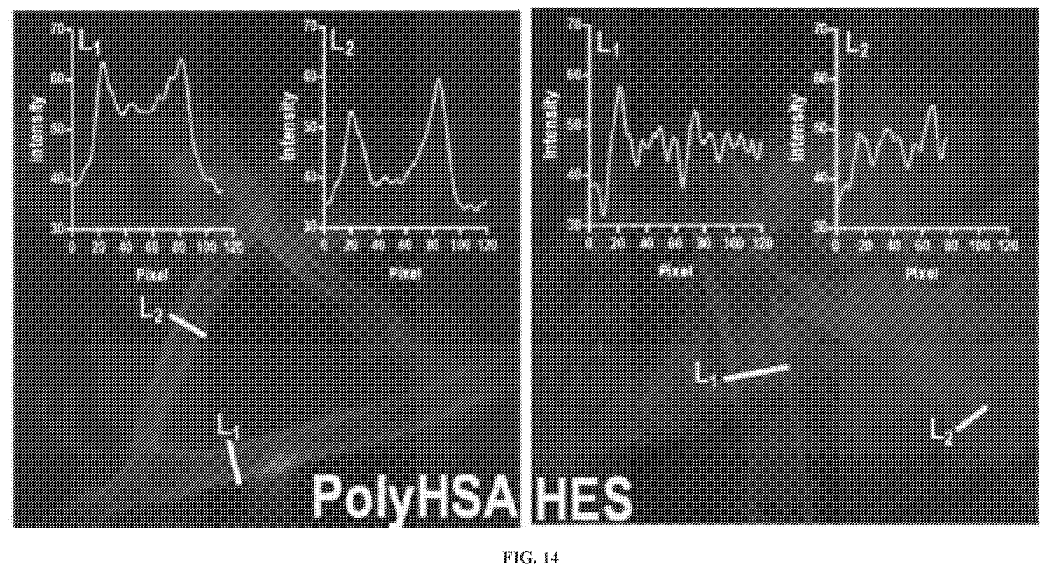

Changes in the microcirculatory hemodynamics in animals that underwent CLP induced septic shock are shown in FIG. 12. At 6 and 12 hours, the relative arteriolar diameter was significantly (P<0.05) expanded in animals resuscitated with PolyHSA compared to animals that received no fluid resuscitation. After 12 hours, the relative arteriole diameter in animals in the HSA treatment group was significantly lower (P<0.05) than animals in the PolyHSA treatment group. Between 2 and 12 hours, the arteriolar blood velocity in animals resuscitated with HSA was significantly lower (P>0.05) than animals that received no fluid resuscitation or animals resuscitated with PolyHSA. In animals in the no resuscitation and PolyHSA treatment group, there was a significant increase in blood velocity compared to baseline conditions. Beginning 2 hours after fluid resuscitation, there was a significant (P<0.05) increase in arteriolar blood flow in animals in the PolyHSA treatment group compared to animals in the HSA treatment group.

At 4 and 6 hours, animals that received no fluid resuscitation had significantly (P<0.05) higher venular blood vessel diameter compared to baseline conditions and animals in the PolyHSA treatment group at the same time point. In all animals, there was a significant (P<0.05) decrease in the venular blood velocity at 2 and 4 hours compared to baseline conditions. This decrease in venular blood velocity was sustained for the remainder of the observation window in animals that received no fluid resuscitation and animals that received HSA. Starting at 6 hours, the venular blood velocity in the PolyHSA treatment group was significantly (P<0.05) greater compared to animals in the HSA and no resuscitation treatment group.

CLP Functional Capillary Density

Changes in the FCD in animals that underwent CLP induced septic shock are shown in FIG. 11C. In animals that received no fluid resuscitation, there was an immediate and sustained (P<0.05) decrease in FCD compared to baseline conditions. Beginning 4 hours after fluid resuscitation, the FCD in animals in the HSA and PolyHSA groups were significantly (P<0.05) greater compared to animals that received no resuscitation. At 12 and 24 hours following fluid resuscitation, animals in the HSA treatment group had significantly (P<0.05) lower FCD compared to baseline conditions and animals in the PolyHSA group at the same timepoint.

CLP Survival

The survival curves for animals that underwent CLP induced septic shock are shown in FIG. 11D. Resuscitation with both HSA and PolyHSA significantly (P<0.05) improved mean survival time compared to animals that received no fluid resuscitation. At four days following CLP, twice the number of animals resuscitated with PolyHSA survived compared to animals in the HSA treatment group.

Discussion

The principal finding of this study was that fluid resuscitation with PolyHSA restores impaired microvascular function after LPS induced endotoxemia and CLP induced polymicrobial sepsis. Overall, fluid resuscitation with the PolyHSA solution resulted in increased normalization of MAP, HR, FCD, and microcirculatory blood flow. When compared to resuscitation with standard HSA, fluid resuscitation with PolyHSA resulted in significantly improved restoration of systemic hemodynamics, microcirculatory hemodynamics, and vascular permeability. Despite observing increases in arteriole diameter and blood flow following resuscitation with PolyHSA, we still observed significant decreases in FCD compared to baseline conditions in LPS induced endotoxemia, but the loss of FCD was attenuated compared to animals that received no fluid resuscitation or unmodified HSA. Unlike arteriole diameter and blood flow, FCD begins to decrease immediately as endotoxemia starts following IV administration of LPS. The continued loss of FCD is likely a result of sustained damage to the endothelial barrier resulting in increased extravascular hydrostatic pressure from extravasation of colloidal proteins. However, in animals resuscitated with PolyHSA, we observed significantly reduced endothelial permeability compared to other treatment groups. Extravasation of PolyHSA is reduced by its increased molecular size, which results in improved maintenance of blood volume, MAP, and capillary pressure, thus preserving FCD. Unlike in LPS induced endotoxemia, animals that underwent CLP induced polymicrobial sepsis had a much slower decay in FCD for the corresponding treatment groups. At around 12 hours, the change in FCD was comparable between animals that underwent LPS induced endotoxemia and CLP induced polymicrobial sepsis. In general, animals resuscitated with HSA had significantly better improvement in the CLP induced polymicrobial sepsis model when compared to the LPS induced endotoxemia model. These significant improvements in FCD following resuscitation with PolyHSA in CLP induced polymicrobial sepsis is likely what leads to the improved survival in the PolyHSA treatment group.

Fluid resuscitation with PolyHSA helps diminish the overactive inflammatory immune response in LPS induced endotoxemia. LPS is recognized by toll-like receptor 4 (TLR4) in all cell-types(24). This leads to a complex inflammatory cascade, and one of the many consequences of this cascade is the classical activation of macrophages (M1 macrophages). These M1 macrophages release TNF-α and other inflammatory cytokines. As we observed in this study, resuscitation with PolyHSA significantly reduced the innate immune response to LPS, evidenced by a significant reduction in pro-inflammatory cytokines. The decrease in pro-apoptotic cytokines (IL-1β, TNF-α) in animals resuscitated with PolyHSA may contribute to the decreased apoptotic cell fraction observed in the tissue. Despite observing decreases in the early-apoptotic cells in the PolyHSA and HSA resuscitation treatment groups, the number of late apoptotic cells was consistent across all groups. The consistent presence of this group of cells likely results from the initial pro-inflammatory response and decreased capillary perfusion. Taken together, these changes in the apoptotic cell fractions indicate a superior recovery in the tissues following resuscitation with PolyHSA.

While resuscitation with PolyHSA did not entirely ameliorate leukocyte adhesion to the endothelium, we still observed significant improvements compared to the no resuscitation and HSA resuscitation groups. This is strong evidence of the preservation of the glycocalyx in animals resuscitated with PolyHSA, as the glycocalyx regulates neutrophil adhesion(25). Despite observing decreased expression of the anti-inflammatory cytokine, IL-10, all measured pro-inflammatory cytokines were suppressed in animals resuscitated with PolyHSA. This temporary decrease in anti-inflammatory responses indicates that resuscitation with PolyHSA may help attenuate the intensity of the initial immune response. 24 hours after LPS administration, anti-inflammatory cytokines were normalized across each treatment group. This restoration of IL-10 at 24 hours indicates that resuscitation with PolyHSA does not have a long term effect on suppressing the anti-inflammatory response.

Overall, fluid resuscitation with PolyHSA resulted in decreases in pro-inflammatory cytokines compared to resuscitation with HSA and no fluid resuscitation. This decrease in pro-inflammatory response may contribute to the improved vascular integrity in the PolyHSA treatment group. Without proper treatment, the immune response, mainly driven by TNF-α, causes the release of reactive oxygen species (ROS), ultimately damaging endothelial cells(26) and causing glycocalyx shedding(27-29). The endothelial glycocalyx plays a vital role in retaining intravascular oncotic pressure by blocking negatively charged proteins, such as HSA, from passing between endothelial cells. HSA flowing into the extravascular area may worsen edema by increasing extravascular oncotic pressure.

Although the decreases in inflammatory cytokines resulting from PolyHSA resuscitation may have an impact on glycocalyx integrity, the increased wall shear stress in the PolyHSA treatment group may facilitate increased glycocalyx regeneration following LPS-induced endotoxemia. Recent studies have demonstrated that glycocalyx production is also regulated by exposure to laminar shear stress(30). In animals that received resuscitation with PolyHSA, the arteriole blood velocity and volumetric flow rate were substantially increased compared to the other two treatment groups. This effect is likely related to the increased circulatory half-life(17) and molecular size of PolyHSA (24 hr, and 410 kDa) compared to unmodified HSA (16 hr and 67 kDa). These effects are likely the cause of the sustained volume expansion following fluid resuscitation with PolyHSA. Given that the viscosity of PolyHSA (4.1 cP) is significantly higher than unmodified HSA (1.5 cP), PolyHSA-enhanced plasma viscosity may have some effect on restoring vascular function due to additional wall shear stress.

One potential mechanism of decreased vascular retention after PolyHSA resuscitation was improved regeneration of the endothelial glycocalyx. Unfortunately, we were unable to observe the glycocalyx structure and shedding during these studies directly. Future studies should include a direct examination of the endothelial glycocalyx throughout treatment with the PolyHSA solution. In addition, the effect of other properties of PolyHSA, such as protein concentration and molecular weight, should be investigated.

Limitations: LPS induced endotoxemia and CLP cannot fully replicate the events that occur during all cases of septic shock, given septic shock's distinct etiologies. However, each model used herein represents a different facet of septic shock, and as such, demonstrates that PolyHSA is efficacious in a variety of conditions. Furthermore, anesthesia has poorly characterized effects on inflammation progression, which may confound results in the partially anesthetized CLP model. Future preclinical studies should examine the effectiveness of PolyHSA in other animal models to confirm that these effects are maintained. Additionally, this study did not examine parameters that directly measure the glycocalyx status, but instead examined the effects of PolyHSA on the glycocalyx's primary physiological role: preservation of vascular permeability. Future studies should more directly examine glycocalyx integrity via fluorescent lectin binding or measure plasma changes in glycocalyx constituents during the progression of septic shock and resuscitation with PolyHSA.

Conclusions

By using a controllable LPS inducible endotoxemia model and a more physiologically relevant CLP induced polymicrobial sepsis model, we found that resuscitation with PolyHSA significantly improves microvascular recovery from septic shock. Overall, the increased M.W. of PolyHSA compared to unmodified HSA played a critical role in maintaining microvascular hemodynamics by interrupting the positive feedback loop in endotoxemia, which stems from glycocalyx disruption. This study also demonstrates that the microvascular response to LPS induced endotoxemia presents more rapidly than macrovascular changes. As such, the development of techniques and instruments to more easily measure the function of microcirculation in the clinic could garner crucial diagnostic information and allow healthcare providers to react to the consequences of impaired microcirculatory function quickly.

Apart from microbial sepsis and LPS induced shock, this data suggests that PolyHSA resuscitation may have a role in the treatment of viral sepsis and secondary bacterial infection associated with COVID-19(31). Infection from SARS-CoV-2 increased vascular permeability via angiotensin-converting enzyme 2 (ACE2) reduction, increased production of ROS by activated neutrophils, and initiation of a cytokine storm(32). A resuscitation fluid with reduced transport across damaged endothelium such as PolyHSA may be beneficial in reducing SARS-CoV-2 associated multiorgan failure.

Abbreviations

SIRS: systemic inflammatory response syndrome, MODS: multiorgan dysfunction syndrome, HSA: human serum albumin, COP: colloidal osmotic pressure, HES: Hydroxyethylene starch, PEG: polyethylene glycol, BSA: bovine serum albumin, FCD: functional capillary density, LPS: lipopolysaccharide, PolyHSA: polymerized HAS, MAP: mean arterial blood pressure, CLP. cecal ligation and puncture, P.I.: propidium iodide, FITC-Dextran: fluorescein isothiocyanate conjugated dextran, TNF-α: tumor necrosis factor-alpha, IL-1α: interleukin 1-alpha, IL-1β: interleukin 1-beta, IL-6: interleukin 6, IL-10: interleukin 10, IL-12: interleukin 12, TLR4: toll-like receptor 4, ROS: reactive oxygen species, ACE2: angiotensin-converting enzyme 2

### REFERENCES

1. Annane P D, Bellissant P E, Cavaillon J M. Septic shock. In: Lancet. Elsevier; 2005. p. 63-78.
2. Angus D C, van der Poll T. Severe Sepsis and Septic Shock. N Engl J Med [Internet]. 2013 Aug. 29 [cited 2020 Jun. 2]; 369(9):840-51. Available from: http://www.nejm.org/doi/10.1056/NEJMra1208623
3. Epstein F H, Parrillo J E. Pathogenetic Mechanisms of Septic Shock [Internet]. Vol. 328, New England Journal of Medicine. Massachusetts Medical Society; 1993 [cited 2020 Jun. 2]. p. 1471-7. Available from: http://www.nejm.org/doi/abs/10.1056/NEJM199305203282008
4. Paoli C J, Reynolds M A, Sinha M, Gitlin M, Crouser E. Epidemiology and costs of sepsis in the United States—an analysis based on timing of diagnosis and severity level. Crit Care Med. 2018; 46(12):1889-97.
5. Angus D C, Linde-Zwirble W T, Lidicker J, Clermont G, Carcillo J, Pinsky M R. Epidemiology of severe sepsis in the United States: Analysis of incidence, outcome, and associated costs of care. Crit Care Med. 2001; 29(7):1303-10.
6. Buras J A, Holzmann B, Sitkovsky M. Animal models of sepsis: Setting the stage. Vol. 4, Nature Reviews Drug Discovery. 2005. p. 854-65.
7. Boerma E C, van der Voort P H J, Ince C. Sublingual microcirculatory flow is impaired by the vasopressin-analogue terlipressin in a patient with catecholamine-resistant septic shock. Acta Anaesthesiol Scand [Internet]. 2005 Oct. 1 [cited 2020 Jun. 2]; 49(9):1387-90. Available from: http://doi.wiley.com/10.1111/j.1399-6576.2005.00752.x
8. Westphal M, Freise H, Kehrel B E, Bone H-G, Van Aken H, Sielenktimper A W. Arginine vasopressin compromises gut mucosal microcirculation in septic rats. Crit Care Med [Internet]. 2004 January [cited 2020 Jun. 2]; 32(1):194-200. Available from: http://journals.lww.com/00003246-200401000-00027
9. van Loon L M, Stolk R F, van der Hoeven J G, Veltink P H, Pickkers P, Lemson J, et al. Effect of Vasopressors on the Macro- and Microcirculation During Systemic Inflammation in Humans In Vivo. SHOCK [Internet]. 2020 Feb. 1 [cited 2020 Jun. 2]; 53(2):171-4. Available from: http://journals.lww.com/10.1097/SHK.0000000000001357
10. Beck V, Chateau D, Bryson G L, Pisipati A, Zanotti S, Parrillo J E, et al. Timing of vasopressor initiation and mortality in septic shock: A cohort study. Crit Care [Internet]. 2014 May 12 [cited 2020 May 17]; 18(3):R97. Available from: http://ccforum.biomedcentral.com/articles/10.1186/cc13868
11. Allen J M, Feild C, Shoulders B R, Voils S A. Recent Updates in the Pharmacological Management of Sepsis and Septic Shock: A Systematic Review Focused on Fluid Resuscitation, Vasopressors, and Corticosteroids. 2019 Apr. 8 [cited 2020 May 17]; 53(4):385-95. Available from: http://journals.sagepub.com/doi/10.1177/1060028018812940
12. Ince C, Mayeux P R, Nguyen T, Gomez H, Kellum J A, Ospina-Tascon G A, et al. The Endothelium in Sepsis [Internet]. Lippincott Williams and Wilkins; Mar. 1, 2016 p. 259-70. Available from: http://content.wkhealth.comAinkback/openurl?sid=WKPTLP:landingpage&an=00024382-201603000-00005
13. Roberts I, Shakur H, Bellomo R, Bion J, Finfer S, Hunt B, et al. Hydroxyethyl starch solutions and patient harm. Vol. 391, The Lancet. Lancet Publishing Group; 2018. p. 736.
14. Wiedermann C J, Eisendle K. Comparison of hydroxyethyl starch regulatory summaries from the Food and Drug Administration and the European Medicines Agency. J Pharm Policy Pract. 2017 Mar. 21; 10(1).
15. Hangai-Hoger N, Nacharaju P, Manjula B N, Cabrales P, Tsai A G, Acharya S A, et al. Microvascular effects following treatment with polyethylene glycol-albumin in lipopolysaccharide-induced endotoxemia. Crit Care Med [Internet]. 2006 January [cited 2020 Apr. 23]; 34(1):108-17. Available from: http://journals.lww.com/00003246-200601000-00016
16. Payne J W. Polymerization of proteins with glutaraldehyde. Soluble molecular weight markers. Biochem J. 1973; 135(4):867-73.
17. Messmer C, Yalcin O, Palmer A F, Cabrales P. Small-volume resuscitation from hemorrhagic shock with polymerized human serum albumin. Am J Emerg Med. 2012; 30(8):1336-46.
18. Elmer J, Cabrales P, Wang Q, Zhang N, Palmer A F. Synthesis and biophysical properties of polymerized human serum albumin. Biotechnol Prog [Internet]. 2011 Jan. 1 [cited 2020 Apr. 23]; 27(1):290-6. Available from: http://doi.wiley.com/10.1002/btpr.531
19. Cabrales P, Tsai A G, Intaglietta M. Microvascular pressure and functional capillary density in extreme hemodilution with low- and high-viscosity dextran and a low-viscosity Hb-based O-2 carrier. Am J Physiol Circ Physiol [Internet]. 2004 July [cited 2018 Dec. 27]; 287(1):H363-73. Available from: http://www.physiology.org/doi/10.1152/ajpheart.01039.2003
20. Hubbard W J, Choudhry M, Schwacha M G, Kerby J D, Rue L W, Bland K I, et al. CECAL LIGATION AND PUNCTURE. Shock [Internet]. 2005 Dec. [cited 2020 Jul. 9]; 24(Supplement 1):52-7. Available from: http://journals.lww.com/00024382-200512001-00009
21. Cuenca A G, Delano M J, Kelly-Scumpia K M, Moldawer L L, Efron P A. Cecal Ligation and Puncture. Curr Protoc Immunol [Internet]. 2010 Nov. 1 [cited 2020 Jul. 9]; 91(1):19.13.1-19.13.11. Available from: https://onlinelibrary.wiley.com/doi/abs/10.1002/0471142735.im1913s91
22. Ortiz D, Briceño JC, Cabrales P. Microhemodynamic parameters quantification from intravital microscopy videos. Physiol Meas [Internet]. 2014 March [cited 2019 Jun. 8]; 35(3):351-67. Available from: http://www.ncbi.nlm.nih.gov/pubmed/24480871
23. Childs E W, Udobi K F, Wood J G, Hunter F A, Smalley D M, Cheung L Y. In Vivo Visualization of Reactive Oxidants and Leukocyte-Endothelial Adherence Following Hemorrhagic Shock. Shock [Internet]. 2002 Nov. 1 [cited 2020 Jun. 2]; 18(5):423-7. Available from: http://journals.lww.com/00024382-200211000-00006
24. Lu Y-C, Yeh W-C, Ohashi P S. LPS/TLR4 signal transduction pathway. Cytokine [Internet]. 2008 May 4 [cited 2020 Jun. 2]; 42(2):145-51. Available from: https://linkinghub.elsevier.com/retrieve/pii/S1043466608000070
25. Schmidt E P, Yang Y, Janssen W J, Gandjeva A, Perez M J, Barthel L, et al. The pulmonary endothelial glycocalyx regulates neutrophil adhesion and lung injury during experimental sepsis. Nat Med. 2012 Aug. 22, 18(8):1217-23.
26. Chen X, Andresen B, Hill M, Zhang J, Booth F, Zhang C. Role of Reactive Oxygen Species in Tumor Necrosis Factor-alpha Induced Endothelial Dysfunction. Curr Hypertens Rev. 2008 Oct. 31; 4(4):245-55.
27. Chappell D, Hofmann-Kiefer K, Jacob M, Rehm M, Briegel J, Welsch U, et al. TNF-α induced shedding of the endothelial glycocalyx is prevented by hydrocortisone and antithrombin. Basic Res Cardiol. 2009; 104(1):78-89.
28. Goligorsky M S, Sun D. Glycocalyx in Endotoxemia and Sepsis. Vol. 190, American Journal of Pathology. Elsevier Inc.; 2020. p. 791-8.
29. Uchimido R, Schmidt E P, Shapiro N I. The glycocalyx: A novel diagnostic and therapeutic target in sepsis [Internet]. Vol. 23, Critical Care. BioMed Central Ltd.; 2019 [cited 2020 Jun. 2]. p. 16. Available from: https://ccforum.biomedcentral.com/articles/10.1186/s13054-018-2292-6
30. Wang G, Kostidis S, Tiemeier G L, Sol WMPJ, de Vries M R, Giera M, et al. Shear Stress Regulation of Endothelial Glycocalyx Structure Is Determined by Glucobiosynthesis. Arterioscler Thromb Vasc Biol [Internet]. 2020 February [cited 2020 Jun. 2]; 40(2):350-64. Available from: https://www.ahajoumals.org/doi/10.1161/ATVBAHA.119.313399
31. Li H, Liu L, Zhang D, Xu J, Dai H, Tang N, et al. SARS-CoV-2 and viral sepsis: observations and hypotheses. Vol. 395, The Lancet. Lancet Publishing Group; 2020. p. 1517-20.
32. Mehta P, McAuley D F, Brown M, Sanchez E, Tattersall R S, Manson J J. COVID-19: consider cytokine storm syndromes and immunosuppression. Vol. 395, The Lancet. Lancet Publishing Group; 2020. p. 1033-4.

### Example 3

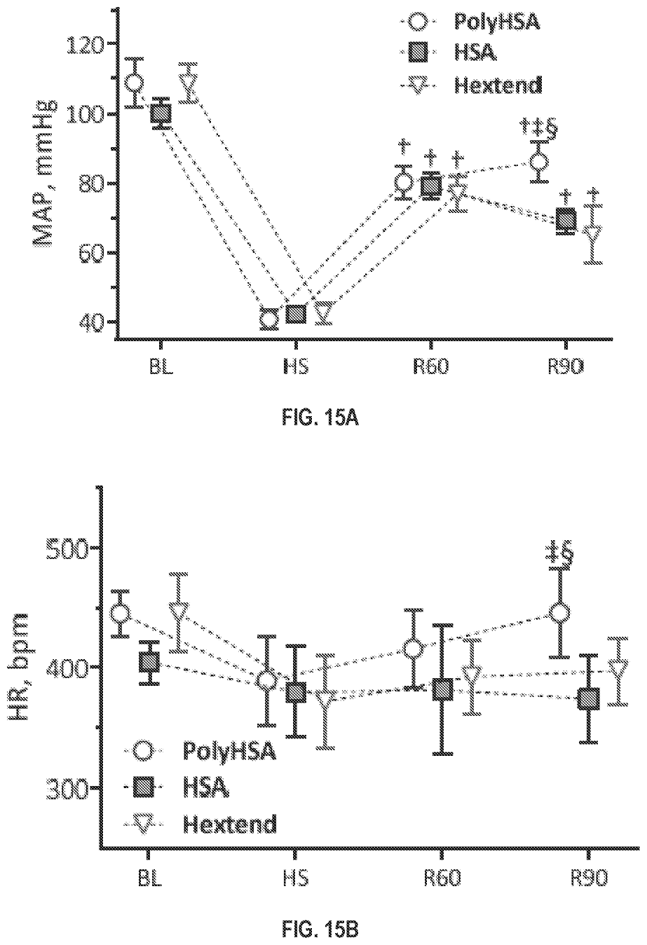

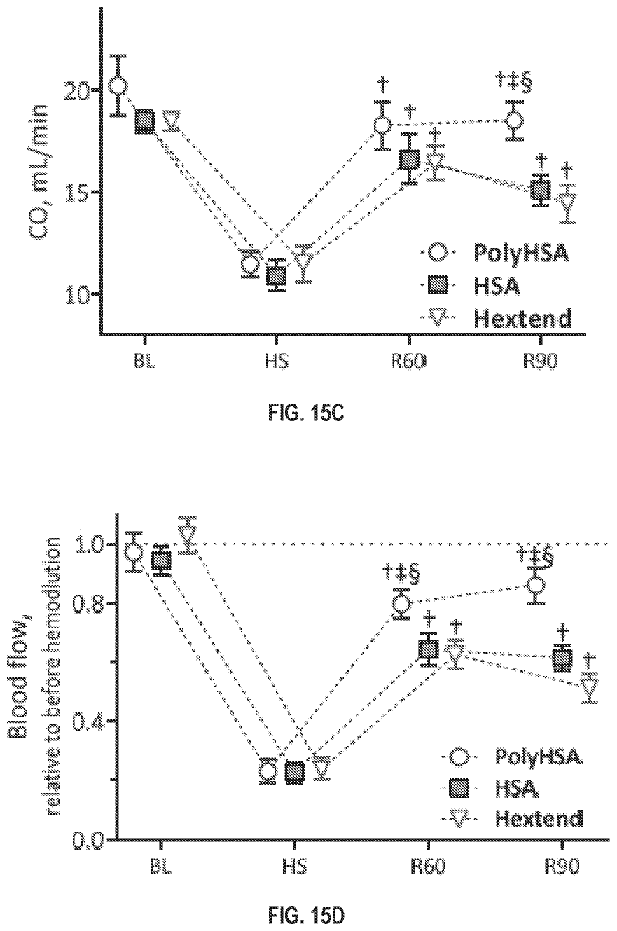

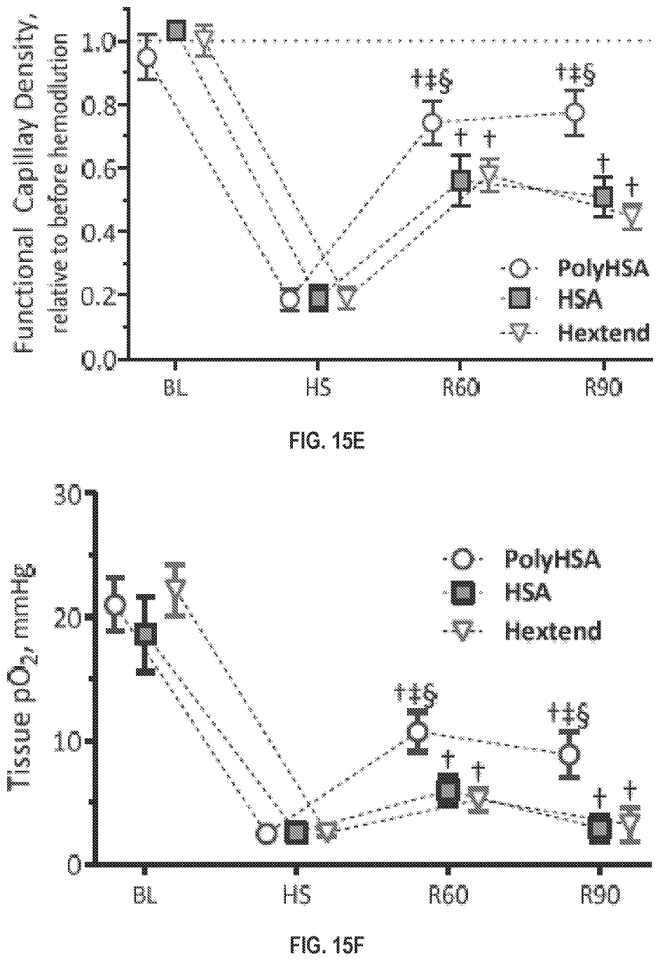

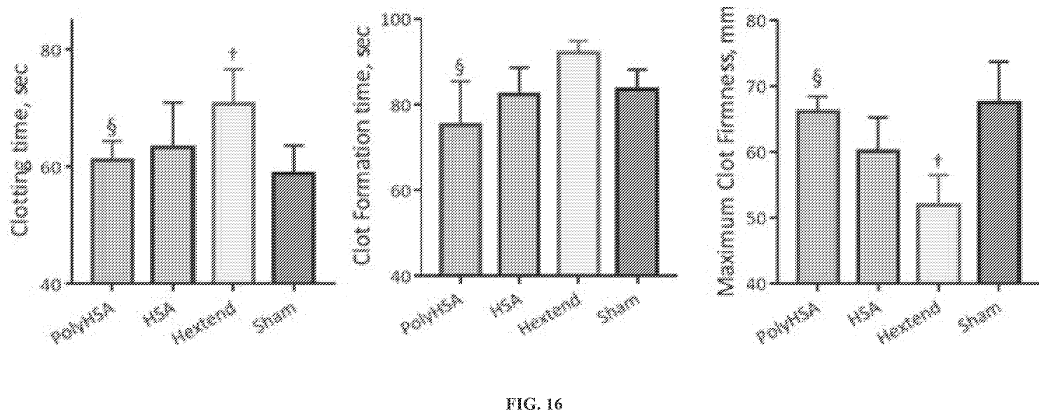

PolyHSA60:1 was studied in a hemorrhagic shock (HS) resuscitation model in hamsters instrumented with the window chamber and compared to Hextend and HSA, FIG. 15. Our preliminary data indicates that resuscitation from HS with PolyHSA60:1 recovers blood pressure, CO, microvascular blood flow, and FCD compared to Hextend and HSA. In additional studies, we compared coagulation after resuscitation from HS in rats with PolyHSA60:1 (at 10 g/dL), Hextend, and HSA (at 10 g/dL), FIG. 16. Animals were subjected to a hemorrhage of 50% of the animal's BV via the femoral artery catheter. Hypovolemia was maintained for 60 minutes before resuscitation. Resuscitation was implemented by infusing the resuscitation fluid until the MAP reached 90% of baseline MAP. If MAP fell below 80% of baseline MAP, additional fluid was infused. Resuscitation with HSA reduced Hct, total protein, fibrinogen, and platelet counts. Clot strength was lower for Hextend compared to HSA and PolyHSA. Thus, PolyHSA preserved the clotting capacity relative to Hextend.

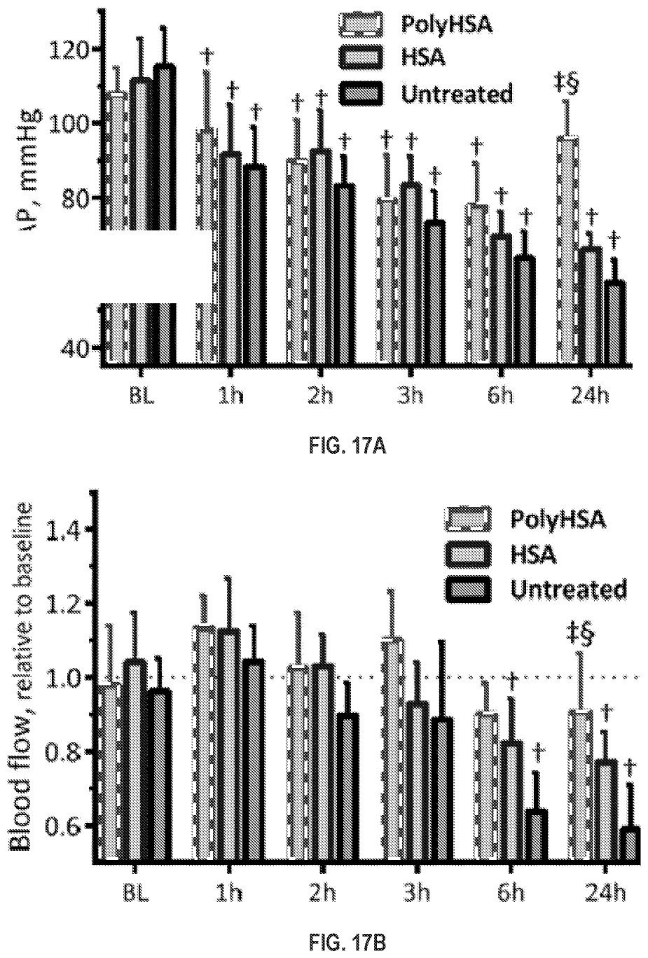

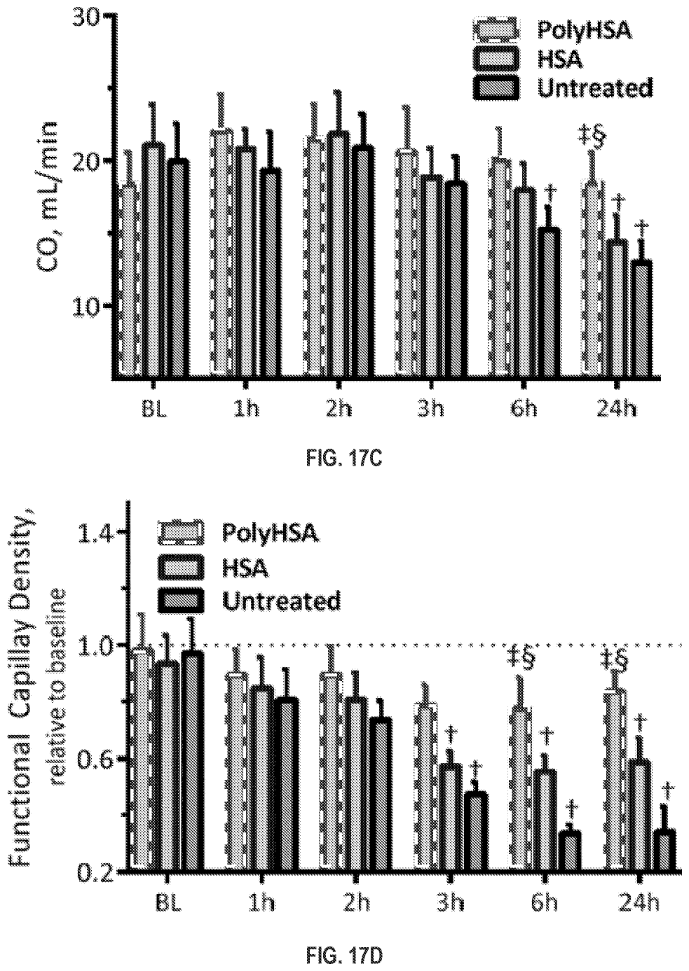

PolyHSA preserves the coupling of systemic and micro hemodynamics during endotoxemia. Our characterization of microvascular function during endotoxemia with plasma substitution using equal volumes of PolyHSA or HSA solutions identified that PolyHSA prevents the decoupling of systemic and microvascular hemodynamics changes (1), FIG. 17. Specifically, 4 hours following LPS injection, a single plasma volume substitution of PolyHSA maintained microvascular hemodynamics (arteriolar blood flow and FCD) and systemic hemodynamics (MAP and CO) in parallel for 24 hours, whereas HSA only preserved systemic hemodynamics, resulting in a precipitous decrease in microvascular hemodynamics as early as 3 hours post volume substitution (1). PolyHSA preserves α-adrenergic receptor response during endotoxemia. Adrenergic drugs are often used to improve blood pressure during septic shock. However, in septic shock, the response to adrenergic drugs is highly attenuated (2). In our preliminary studies, plasma volume substitution with PolyHSA hours after LPS injection, resulted in a preserved arteriolar response to phenylephrine, whereas plasma volume substitution with HSA resulted in an 80% reduction in the arteriolar diameter response to phenylephrine compared to baseline. Similarly, the MAP response to phenylephrine was preserved with PolyHSA, whereas the HSA group experienced a 15% reduction in the MAP response compared to baseline.

PolyHSA reduces cardiac dysfunction during endotoxemia. Systemic inflammation eventually results in cardiac failure (3). During endotoxemia, LV function is compromised, and CO is reduced, despite decreased afterload. Results demonstrate that infusion of PolyHSA after LPS injection results in preserved preload and intravascular volume, reduced edema, and decreased acute lung injury compared to HSA. Infusion of PolyHSA during systemic inflammation restored cardiac function, and improved hemodynamics, preventing MODS. PolyHSA preserves microvascular perfusion during endotoxemia. Systemic hemodynamics fail to report microcirculatory deficits; and if left uncorrected, microcirculatory dysfunction can lead to organ dysfunction.

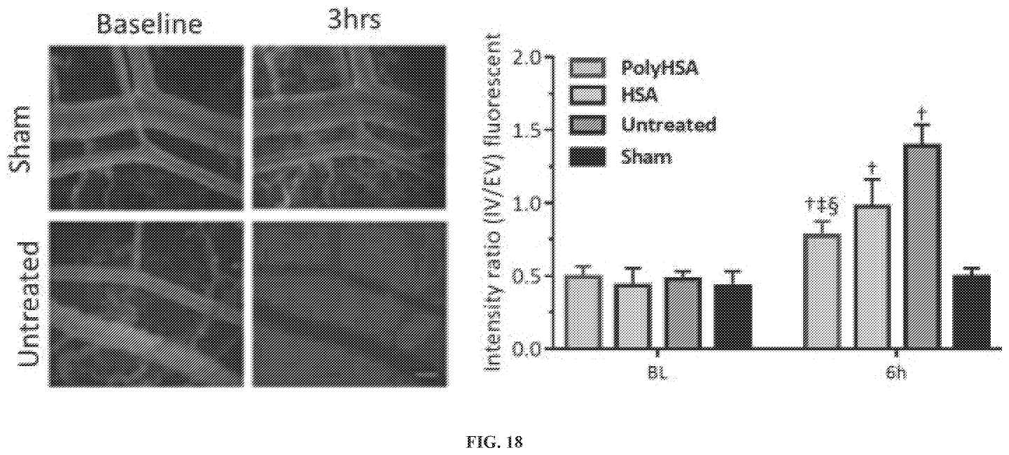

Studies show that plasma substitution with PolyHSA during endotoxemia preserves microvascular perfusion compared to HSA, FIG. 17. FCD, capillary Hct, and blood flow are impaired within minutes after LPS injection, and plasma volume substitution with PolyHSA maintains FCD, capillary Hct, and blood flow by preventing fluid extravasation. PolyHSA reduced cytokine response during endotoxemia. During Systemic inflammatory response syndrome (SIRS), the innate immune system responds by using pattern Toll-like receptors (TLRs) to recognize pathogen-associated molecular patterns. Surface molecules of gram-positive and gram-negative bacteria (peptidoglycans and lipopolysaccharides) bind to TLR-2 and TLR-4, respectively. Their binding initiates an intracellular signaling cascade that culminates in nuclear transport of the transcription factor nuclear factor kappa B (NFκB), which triggers the transcription of TNF a and IL-6. PolyHSA reduced the accumulation of inflammatory cytokines (FIG. 9C-9H), which regulate adhesion and neutrophils activation. Thus, PolyHSA could restore microvascular blood flow, reduce cytokine buildup and fluid extravasation, limiting MODS. PolyHSA prevented changes in microvascular permeability consistent with preservation of FCD. A hallmark of SIRS, sepsis, and septic shock is an increase in vascular permeability, stimulated in part by the accumulation of pro-inflammatory cytokines (4). Thus, plasma substitution with PolyHSA could prevent protein extravasation and reduction in BV. Our results show that PolyHSA preserves vascular permeability after LPS, FIG. 18 (1).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

### REFERENCES

1. Belcher D A, Williams A T, Palmer A F, Cabrales P. Polymerized albumin restores impaired hemodynamics in endotoxemia and polymicrobial sepsis. Sci Rep. 2021; 11(1):10834. Epub 2021/05/27.
2. Pollard S, Edwin S B, Alaniz C. Vasopressor and inotropic management of patients with septic shock. Pharmacy and Therapeutics. 2015; 40(7):438.
3. Smeding L, Plotz F B, Groeneveld A B, Kneyber M C. Structural changes of the heart during severe sepsis or septic shock. Shock. 2012; 37(5):449-56.
4. Chong D, Sriskandan S. Pro-inflammatory mechanisms in sepsis 2011.

1. A method of treating hypercytokinemia in a subject in need thereof, the method comprising:
administering to the subject a therapeutically effective amount of PolyHSA to reduce circulating cytokine levels by at least 5%, such as from 5% to 70%.

2. The method of claim 1, wherein the hypercytokinemia is induced by an infectious agent such as influenza (e.g., H1N1 influenza or H5N1 influenza), coronavirus infection (e.g., avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza B, Parainfluenza virus, Ebola, Epstein-Barr virus, cytomegalovirus, or group A streptococcus.

3. The method of claim 1, wherein the hypercytokinemia is associated with graft-versus-host disease.

4. A method of preventing hypercytokinemia in a subject, the method comprising:
administering to the subject a therapeutically effective amount PolyHSA to reduce or prevent an increase in circulating cytokine levels.

5. The method of claim 4, wherein the subject is infected with or has been exposed to an infectious agent such as influenza (e.g., H1N1 influenza or H5N1 influenza), coronavirus infection (e.g., avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza B, Parainfluenza virus, Ebola, Epstein-Barr virus, cytomegalovirus, or group A streptococcus.

6. The method of claim 4, wherein the subject has received or will receive transplanted cells, transplanted tissue, a transplanted organ, or any combination thereof.

7. The method of claim 6, wherein the transplanted cells, transplanted tissue, a transplanted organ, or any combination thereof comprise an allograft or a xenograft.

8. A method of treating endothelial dysfunction in a subject, the method comprising:
administering to the subject a therapeutically effective amount of PolyHSA to reduce circulating levels of a biomarker for endothelial dysfunction in the subject.

9. (canceled)

10. (canceled)

11. The method of claim 8, wherein the biomarker for endothelial dysfunction comprises syndecan-1.

12. A method of treating endothelial dysfunction in a subject in need thereof, the method comprising:
administering to the subject a therapeutically effective amount of PolyHSA to reduce endothelial barrier permeability.

13. (canceled)

14. The method of claim 1, wherein the subject has a normal blood pressure.

15. The method of claim 1, wherein the PolyHSA is administered via infusion or exchange transfusion.

16. The method of claim 1, wherein the PolyHSA is administered via infusion.

17. The method of claim 16, wherein the infusion comprises infusion of a volume of a composition comprising the PolyHSA, and
wherein the volume comprises from 10% to 30% of the subject's total blood volume.

18. The method of claim 1, wherein the PolyHSA is administered via exchange transfusion.

19. The method of claim 18, wherein the exchange transfusion comprises exchange transfusion of from 5% to 50% of the subject's total blood volume with a composition comprising the PolyHSA.

20. The method of claim 1, wherein the PolyHSA is administered in an amount effective to reduce circulating cytokine levels by at least 5%, such as from 5% to 70%.

21. The method of claim 1, wherein the PolyHSA is administered in a therapeutically effective amount to reduce an immune response.

22. The method of claim 1, wherein the PolyHSA is administered in a therapeutically effective amount to reduce the number of leukocytes adhered to endothelial tissue in the subject.

23. The method of claim 1, wherein the PolyHSA is administered in a therapeutically effective amount to improve vascular integrity.

24. The method of claim 1, wherein the PolyHSA has a molecular weight ranging from 100 kDa to 50,000 kDa, such as from 100 kDa to 300 kDa, from 100 kDa to 500 kDa, from 100 kDa to 750 kDa, from 300 kDa to 500 kDa, from 300 kDa to 750 kDa, or from 500 kDa to 750 kDa.
